SEQUENCE LISTING (36 sequences)

SEQ ID NO: 1 - DNA, 76 bp, synthetic construct
  source 1..76: mol_type = other DNA
caggaacccctagtgatggagttggagttggctgcgcgctcgctcgctcactgctgcgcgctcgctcgctcactga

SEQ ID NO: 2 - DNA, 84 bp, synthetic construct
  source 1..84: mol_type = other DNA
cgcgtcagtgagcgagcgagcgcgcagcagtgagcgagcgagcgcgcagccaactccaactccatcactaggggttcctggtac

SEQ ID NO: 3 - DNA, 80 bp, synthetic construct
  source 1..80: mol_type = other DNA
gaccgcagtgagcgagcgagcgcgcagcagtgagcgagcgagcgcgcagccaactccaactccatcactaggggttccta

SEQ ID NO: 4 - DNA, 81 bp, synthetic construct
  source 1..81: mol_type = other DNA
agcttaggaacccctagtgatggagttggagttggctgcgcgctcgctcgctcactgctgcgcgctcgctcgctcactgcg

SEQ ID NO: 5 - DNA, 33 bp, synthetic construct
  source 1..33: mol_type = other DNA
tcactgacgcgtcctgaacgacctgaacaatct

SEQ ID NO: 6 - DNA, 33 bp, synthetic construct
  source 1..33: mol_type = other DNA
aactagacgcgtatccttagcgagggggcaggg

SEQ ID NO: 7 - DNA, 34 bp, synthetic construct
  source 1..34: mol_type = other DNA
tttgtaggtaaccaaccgtaatccggacctcacc

SEQ ID NO: 8 - DNA, 34 bp, synthetic construct
  source 1..34: mol_type = other DNA
gcacgtggttaccagggtacaaggcagggcctgc

SEQ ID NO: 9 - DNA, 33 bp, synthetic construct
  source 1..33: mol_type = other DNA
tcgaacatcgatatggaagatgccaaaaacatt

SEQ ID NO: 10 - DNA, 33 bp, synthetic construct
  source 1..33: mol_type = other DNA
agatctctcgagttacacggcgatcttgccgcc

SEQ ID NO: 11 - DNA, 34 bp, synthetic construct
  source 1..34: mol_type = other DNA
tcactgacgcgtggctgttcaaagaggccaacgt

SEQ ID NO: 12 - DNA, 34 bp, synthetic construct
  source 1..34: mol_type = other DNA
aactagacgcgttccaactttgtcccagcgccga

SEQ ID NO: 13 - DNA, 34 bp, synthetic construct
  source 1..34: mol_type = other DNA
tttgtaggtaacctgccagctgaagtcagagctg

SEQ ID NO: 14 - DNA, 34 bp, synthetic construct
  source 1..34: mol_type = other DNA
gcacgtggttaccttctggggtgggatcaggata

SEQ ID NO: 15 - DNA, 27 bp, synthetic construct
  source 1..27: mol_type = other DNA
gaccgtaatctctagattgaggaacaa

SEQ ID NO: 16 - DNA, 28 bp, synthetic construct
  source 1..28: mol_type = other DNA
agctttgttcctcaatctagagattacg

SEQ ID NO: 17 - DNA, 33 bp, synthetic construct
  source 1..33: mol_type = other DNA
tcgaacgaattcatggactataaggaccacgac

SEQ ID NO: 18 - DNA, 33 bp, synthetic construct
  source 1..33: mol_type = other DNA
agatctctcgagtcactttttcttttttgcctg

SEQ ID NO: 19 - DNA, 36 bp, synthetic construct
  source 1..36: mol_type = other DNA
tttgtaggtaaccagtttggttaattaacgtaccga

SEQ ID NO: 20 - DNA, 36 bp, synthetic construct
  source 1..36: mol_type = other DNA
gcacgtggttaccgtaagttatgtaacgtaattcaa

SEQ ID NO: 21 - DNA, 33 bp, synthetic construct
  source 1..33: mol_type = other DNA
tctcccggtaccatgatcaagttccggggccac

SEQ ID NO: 22 - DNA, 33 bp, synthetic construct
  source 1..33: mol_type = other DNA
catggtgctagcgtaagttatgtaacgtaattc

SEQ ID NO: 23 - DNA, 21 bp, synthetic construct
  source 1..21: mol_type = other DNA
tccgcgttacataacttacgg

SEQ ID NO: 24 - DNA, 20 bp, synthetic construct
  source 1..20: mol_type = other DNA
gggcgtacttggcatatgat

SEQ ID NO: 25 - DNA, 21 bp, synthetic construct
  source 1..21: mol_type = other DNA
cctgggtggtcttccctgatc

SEQ ID NO: 26 - DNA, 25 bp, synthetic construct
  source 1..25: mol_type = other DNA
gcaccttgtcacaaagcagataaac

SEQ ID NO: 27 - DNA, 26 bp, synthetic construct
  source 1..26: mol_type = other DNA
tgatcacaatgctactatgcccttgg

SEQ ID NO: 28 - DNA, 26 bp, synthetic construct
  source 1..26: mol_type = other DNA
ctgctgtcttgtccattcgagaaatc

SEQ ID NO: 29 - DNA, 8412 bp, synthetic construct
  source 1..8412: mol_type = other DNA
aggaacccctagtgatggagttggagttggctgcgcgctcgctcgctcactgctgcgcgctcgctcgctcactgacgcgtgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcactgacagccctgctgctgcctctggctctgctgctgcatgccgctagacccgattacaaggatgacgacgataagggtggtggtggttctatccagatgacacagactacatcctccctgtctgcctctctgggagacagagtcaccatcagttgcagggcaagtcaggacattagcaattttttaaactggtatcagcagaaaccagatggaactgttaaactcctgatctacgcgacatcaagattacattctggagtcccatcaaggttcagtggcagtgggtctggaacagatttttctctcaccattagcaaactggagcaagaagatattgccacttacttttgccaacagggtaatacgcttccacttacgttcggcgctgggacaaagttggaacttaaaggtggtggtggttctggcggcggcggctccggaggaggaggatcgctgcaacagtctggacctgagttggtgaagcctggggcttcagtgaagatttcctgcaaggcttctggatacacattcactgactactacatgaattgggtgaagcttagccatggaaagagccttgagtggattggagatattgttcctaacaatggtgatactacttacaaccagaatttcagaggcaaggccacattgactgtagacaagtcctccagcacagcctacatggagctccgcagcctgacatctgaggactctgcagtctattactgtgcaagattcagtaattacgtttacccttttgactactggggccaaggcaccactatcacagtctccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaaggatacgcgtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattccccggggatccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccaaccgtaatccggacctcacccagctccctggtggctttcatcaactctcgctgtacatctccgggcggttcctacggccatctctccattggtaccatgagcccttctttaggattcccacctcagatgagtcatcaaaaaggaacttcacctccctatggagtccagccctgtgtaccacatgactctactcggggttcaatgatgcttcacccccagtcccggggaccacgtgcaacctgccagctgaagtcagagctggatatgatggttggcaagtgcccggaggaccctttggaaggggacatgtctagccccaactccacaggcacacaggatcacctgttggggatgctggatgggcgggaggacctggagagagaggagaagcctgagcctgagtctgtgtatgagacagactgccgctgggatggttgcagccaggagttcgattcccaggagcagctggtgcaccacatcaacagtgagcatatccacggggagcggaaggaattcgtgtgccattggggaggttgctccagggagctgaggcccttcaaggcccaatacatgctggtggtgcacatgcgcagacacacgggcgagaagccacacaagtgcacgtttgaaggctgtcggaagtcctattcacgccttgaaaacctcaagacgcaccttcggtcgcacacgggtgagaagccttacatgtgtgagcaagaaggttgcagcaaggcctttagcaatgccagtgaccgcgccaagcaccagaatcggacccactccaatgagaagccatacgtgtgcaagctccccggctgcaccaagcgctacacagatcccagctcgctccgcaaacacgtgaagacagtgcatggtccggatgcccacgtgaccaagcggcatcgaggggatggccccttgccacgggctcagcccctctccacagtggagcccaagcgggaaagggaaggaggatccggcagggaagagagcagactgactgtgcccgagagtgccatgccgcagcagagccccggagcgcagtcctcttgcagcagcgaccactccccagcaggcagtgcggccaacacggacagcggcgtggagatggccggcaacgccgggggcagcactgaggacttgtccagcttggatgaaggaccttgtgtctcggccaccggactctccacgcttcgccgcctggagaaccttaggctggatcagctgcatcagctccggcccatagggtctcggggtctcaaactgcccagcttaacccacgctggcgcacctgtgtctcgccgtctgggccccccagtctccctggaccgccgcagcagcagctccagcagcatgagctctgcttacacagtcagccgcaggtcctccctggcatcccctttcccgccgggaaccccaccagagaatggggcatcgtcactacctggcctcacacctgctcagcactacatgctccgtgccagatatgcttcagccagggggagtggcaccccgcccactgcagctcacagcctggatcggatgggaggtctttctgttcctccttggagaagccgaaccgagtacccgggatacaacccaaatgcaggggtcactcggagggccagtgacccagcccgggctgctgaccacccagctccagccagagtccagcggttcaagagcctgggatgtgtccacacgccccctagtgtggcaacgggacggaacttcgatccccaccaccctacctctgtctattcgccacagccccccagcatcaccgaaaatgttgccatggatactagggggctacaggaggagccagaggttggaacttctgtgatgggcaatggtctgaacccatacatggatttttcctccactgatactctgggatatgggggacccgaggggacggcagctgagccttatgaagctaggggtccaggttccctgcctcttgggcctggtccaccaaccaactatggccctggccactgtgcccagcaggtctcctatcctgatcccaccccagaaaactggggtgagttcccttctcatgctggggtgtaccctagcaataaggctccgggtgctgcctatagccagtgtcctcgacttgagcattatggacaagtgcaggtaaaaccagaacaagggtgcccagtggggtctgactccaccggattggcaccctgcctcaatgcccaccccagtgaagggtccccaggcccgcagcctctgttttcacatcatccccagctccctcagccccagtatccccagtcgggtccctatcctcagcctccccatggttatctctcaacagaacccaggcttggcctcaatttcaacccctcctcctctcattccacaggacagctcaaagctcagctggtgtgtaattacgttcagtcgcagcaggaattgttgtgggagggaagaaaccggggagggctccccaaccaggaactcccataccagagccccaagtttctggggggttcccaagttagtcagagccctgccaagaccccagcagcagcggcggcagcatatggatctggctttgcacctgcttcggccaatcacaaatcaggctcctatcctgccccttcaccctgccatgaaactttcaccgtgggagtaaacaggccttcccacaggccagcagcaccaccccgacttctgcccccgctgtccccttgctatgggcccctcaaggtgggggataccaaccccagctgtggccatcctgaggtgggcaggttaggagcaggccctgccttgtaccctggtaaccacgtg

SEQ ID NO: 30 - DNA, 7625 bp, synthetic construct
  source 1..7625: mol_type = other DNA
aggaacccctagtgatggagttggagttggctgcgcgctcgctcgctcactgctgcgcgctcgctcgctcactgacgcgtggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcactgacagccctgctgctgcctctggctctgctgctgcatgccgctagacccgattacaaggatgacgacgataagggtggtggtggttctatccagatgacacagactacatcctccctgtctgcctctctgggagacagagtcaccatcagttgcagggcaagtcaggacattagcaattttttaaactggtatcagcagaaaccagatggaactgttaaactcctgatctacgcgacatcaagattacattctggagtcccatcaaggttcagtggcagtgggtctggaacagatttttctctcaccattagcaaactggagcaagaagatattgccacttacttttgccaacagggtaatacgcttccacttacgttcggcgctgggacaaagttggaacgcgtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgatatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaacctgccagctgaagtcagagctggatatgatggttggcaagtgcccggaggaccctttggaaggggacatgtctagccccaactccacaggcacacaggatcacctgttggggatgctggatgggcgggaggacctggagagagaggagaagcctgagcctgagtctgtgtatgagacagactgccgctgggatggttgcagccaggagttcgattcccaggagcagctggtgcaccacatcaacagtgagcatatccacggggagcggaaggaattcgtgtgccattggggaggttgctccagggagctgaggcccttcaaggcccaatacatgctggtggtgcacatgcgcagacacacgggcgagaagccacacaagtgcacgtttgaaggctgtcggaagtcctattcacgccttgaaaacctcaagacgcaccttcggtcgcacacgggtgagaagccttacatgtgtgagcaagaaggttgcagcaaggcctttagcaatgccagtgaccgcgccaagcaccagaatcggacccactccaatgagaagccatacgtgtgcaagctccccggctgcaccaagcgctacacagatcccagctcgctccgcaaacacgtgaagacagtgcatggtccggatgcccacgtgaccaagcggcatcgaggggatggccccttgccacgggctcagcccctctccacagtggagcccaagcgggaaagggaaggaggatccggcagggaagagagcagactgactgtgcccgagagtgccatgccgcagcagagccccggagcgcagtcctcttgcagcagcgaccactccccagcaggcagtgcggccaacacggacagcggcgtggagatggccggcaacgccgggggcagcactgaggacttgtccagcttggatgaaggaccttgtgtctcggccaccggactctccacgcttcgccgcctggagaaccttaggctggatcagctgcatcagctccggcccatagggtctcggggtctcaaactgcccagcttaacccacgctggcgcacctgtgtctcgccgtctgggccccccagtctccctggaccgccgcagcagcagctccagcagcatgagctctgcttacacagtcagccgcaggtcctccctggcatcccctttcccgccgggaaccccaccagagaatggggcatcgtcactacctggcctcacacctgctcagcactacatgctccgtgccagatatgcttcagccagggggagtggcaccccgcccactgcagctcacagcctggatcggatgggaggtctttctgttcctccttggagaagccgaaccgagtacccgggatacaacccaaatgcaggggtcactcggagggccagtgacccagcccgggctgctgaccacccagctccagccagagtccagcggttcaagagcctgggatgtgtccacacgccccctagtgtggcaacgggacggaacttcgatccccaccaccctacctctgtctattcgccacagccccccagcatcaccgaaaatgttgccatggatactagggggctacaggaggagccagaggttggaacttctgtgatgggcaatggtctgaacccatacatggatttttcctccactgatactctgggatatgggggacccgaggggacggcagctgagccttatgaagctaggggtccaggttccctgcctcttgggcctggtccaccaaccaactatggccctggccactgtgcccagcaggtctcctatcctgatcccaccccagaaggtaaccacgtg

SEQ ID NO: 31 - DNA, 6781 bp, synthetic construct
  source 1..6781: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattcatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccagtttggttaattaacgtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcgagggaccagccaagatcggttttagagctagaaatagcaagttaaaataaggctagtccgttatcaacttgaaaaagtggcaccgagtcggtgctttttttacggatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgagactgtgggcatgagcacggttttagagctagaaatagcaagttaaaataaggctagtccgttatcaacttgaaaaagtggcaccgagtcggtgcttttttgaattacgttacataacttacggtaaccacgtg

SEQ ID NO: 32 - DNA, 145 bp, synthetic construct
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 33 - DNA, 74 bp, synthetic construct
  source 1..74: mol_type = other DNA
aggaacccctagtgatggagttggagttggctgcgcgctcgctcgctcactgctgcgcgctcgctcgctcactg

SEQ ID NO: 34 - DNA, 73 bp, synthetic construct
  source 1..73: mol_type = other DNA
aggaacccctagtgatggagttggccactccctctctgcgcgctcgctcgctcactgaggccgggcgaccaaa

SEQ ID NO: 35 - DNA, 20 bp, synthetic construct
  source 1..20: mol_type = other DNA
aggaacccctagtgatggag

SEQ ID NO: 36 - DNA, 22 bp, synthetic construct
  source 1..22: mol_type = other DNA
ctgcgcgctcgctcgctcactg

(19) United States
(12) Patent Application Publication
(10) Pub. No.: US 2026/0357294 A1
(43) Pub. Date: Nov. 12, 2026

(54) Construction, Production, and Use of a Recombinant Adeno-Associated Virus Vector for Large Gene Packaging and Expression

(71) Applicant: SUZHOU GENEHEALTH BIOTECHNOLOGY, CO.LTD., SUZHOU, JIANGSU, CHINA, Suzhou, Jiangsu (CN)

(72) Inventors: Jiahong SHAO, Suzhou (CN); Xiaoming ZHAO, Suzhou (CN); Xiang WU, Suzhou (CN); Pengcheng TAN, Suzhou (CN); Tingjun XUN, Suzhou (CN); Zhenzhen LEI, Suzhou (CN); Yang LU, Suzhou (CN)

(73) Assignee: SUZHOU GENEHEALTH BIOTECHNOLOGY, CO.LTD., Suzhou (CN)

(21) Appl. No.: 19/148,320

(22) PCT Filed: Nov. 13, 2023
(86) PCT No.: PCT/CN2023/131178

§ 371 Date: Jun. 9, 2026

(30) Foreign Application Priority Data
Jan. 16, 2023 (CN) 202310055399.2

(51) Int. Cl. C12N 15/86

(52) U.S. Cl. CPC C12N 15/86; C12N 2750/14143; C12N 2750/14152; C12N 2830/50

(57) ABSTRACT

A construction system of recombinant adeno-associated virus vectors for packaging and expressing large genes includes a first packaging and expression vector and a second packaging and expression vector. From 5′ end to 3′ end, the first packaging and expression vector includes a first ITR element or analogous element and partial DNA of a first expression cassette including a promoter, a target gene, and a poly A signal of a positive strand. From 5′ end to 3′ end, the second packaging and expression vector includes a second ITR element or analogous element and partial DNA of a second expression cassette including a poly A signal, a target gene, and a promoter of a negative strand. The positive and negative strands undergo partial complementary base pairing after being transduced into host cells at 3′ ends to synthesize a second strand, thereby forming a complete expression cassette of the target gene.

### FIELD OF TECHNOLOGY

The present disclosure relates to the technical field of gene vectors, gene therapy, and gene editing, particularly to the construction, production, and use of a recombinant adeno-associated virus vector for large gene packaging and expression.

### BACKGROUND

Gene therapy targets patients with genetic mutations caused by abnormal gene expression profiles. It includes the treatment or prevention of genetic diseases caused by gene defects, dysregulation, or aberrant expression, such as diseases resulting from underexpression or overexpression of genes, or malignant tumors by delivering corrective genetic material to the patient to treat, prevent, or alleviate such conditions. Current gene delivery vectors mainly include non-viral vectors and viral gene delivery vectors. Among the many available viral-derived gene vectors, recombinant retroviruses, recombinant lentiviruses, and recombinant adenoviruses etc., especially recombinant adeno-associated virus (rAAV) vectors are becoming increasingly popular.

Adeno-associated virus (AAV) belongs to the Parvoviridae family. Due to its advantages, such as non-pathogenicity, low immunogenicity, and broad host tropism, AAV has become the most promising gene vector for gene therapy. AAV is a non-enveloped single-stranded DNA virus, carrying a linear single-stranded DNA genome of 4.6~4.7 kb. Both ends of the AAV DNA genome are provided with inverted terminal repeats (ITRs), each about 145 base pairs in length. The terminal 125 base pairs form a long palindromic structure, capable of self-complementary base pairing, which allows the ITR to fold into a T-shaped hairpin structure. Positive strand and negative strand DNA genomes carrying the wild-type ITRs can be packaged into the AAV capsid with equal probability, resulting in equal numbers of positive or negative strand AAV virions. Upon infecting host cells, AAV enters the nucleus, where uncoating occurs. After uncoating, the ITRs at the genomic termini fold to form the T-shaped palindromic structure, and a second strand is then synthesized using the 3′ end as a primer, forming a double-stranded molecule that subsequently initiates gene expression. Alternatively, the AAV DNA positive strand and negative strand genomes can also form a double-stranded DNA molecule through complementary base pairing, thereby initiating gene expression. Furthermore, intermolecular interactions between ITRs lead to the spontaneous formation of dimers and higher-order concatemers, allowing such molecules to achieve long-term, even lifelong, sustained expression of exogenous genes within host cells.

The rAAV vector has a limited packaging capacity of approximately 4.7 kb, which restricts the application of AAV-based gene therapy for diseases caused by large genes. Current primary strategies for enabling AAV-mediated delivery of large genes are as follows. The first strategy is cis-splicing, which exploits the homologous recombination of ITR sequences, thereby linking multiple AAV genomes together. Specifically, one AAV vector carries the promoter, the 5′ portion of the coding region, and the splice donor signal, and the other AAV vector carries the splice acceptor signal, the remaining portion of the coding region, and the polyadenylation (polyA) signal. When the two AAV genomes concatenate in a head-to-tail orientation to form concatemers, the redundant ITR sequences are removed via splicing to generate a complete gene expression cassette.

The second strategy involves that two AAV vectors carrying homologous coding gene sequences undergo homologous recombination in the host cells to form a complete expression cassette for the large gene.

The third strategy is a hybrid dual-vector approach, combining the aforementioned first and second strategies. This strategy takes advantage of the concatemerization activity of AAV genomes, whereby independent AAV vector genomes are brought together via recombination. In this case, the original genome is intentionally divided into a left half and a right half, with one carrying 5′ splicing element and the other one carrying 3′ splicing element, respectively, followed by splicing to ensure correct transgenic protein production post-infection. One study demonstrated that inserting an 872 bp highly recombinogenic alkaline phosphatase sequence into AAV vectors, which contain intron splicing sites, may result in high-level, transgenic-independent homologous recombination of the alkaline phosphatase sequence in vivo after administration of the split vectors. This third strategy may increase the expression of full-length functional proteins.

The fourth strategy involves constructing the full-length gene in the same packaging plasmid, such that during viral packaging, a population of AAV virions carrying a truncated gene fragment at different positions is generated. Specifically, the resulting AAV vectors include truncated genes of varying lengths. After transducing these AAV vectors, a complete gene expression cassette is produced via annealing of complementary regions across different AAV vector genomes using a single-stranded template followed by DNA synthesis.

The fifth strategy utilizes intein-mediated protein trans-splicing technology, enabling the design of split AAV vectors to package larger AAV genomes. Similar to intron-mediated RNA splicing, inteins catalyze protein splicing and facilitate precise ligation of two separate polypeptides via trans-splicing. When delivering larger gene expression cassettes using this technology, multiple AAV vectors (each encoding one fragment of the target protein flanked by short split inteins) are delivered to the same cell. Protein trans-splicing then occurs to form the full-length protein. This method has been successfully applied in animals for delivering dystrophin, FVIII, CFTR, CRISPR-Cas9, ABCA4, and the 290 kDa centrosomal protein (CEP290).

In summary, current AAV-based methods for large-gene expression exhibit low expression efficiency, poor stability, and unclear mechanisms, and thus cannot be effectively applied for AAV-based gene therapy of diseases caused by large genes.

### SUMMARY

The present disclosure provides a construction, production, and use of a recombinant adeno-associated virus vector for large gene packaging and expression to enhance large gene expression efficiency.

The present disclosure specifically adopts the following technical solutions.

The first aspect of the present disclosure provides a construction system of a recombinant adeno-associated virus vector for large gene packaging and expression, wherein the construction system enables packaging and expression of a DNA with a length exceeding 4.7 kb. The construction system includes a first packaging and expression vector and a second packaging and expression vector.

From 5′ end to 3′ end, the first packaging and expression vector includes a first ITR element or analogous element and a first expression cassette. The first expression cassette includes a promoter, a target gene, and a polyadenylation (polyA) signal of a positive strand.

From 5′ end to 3′ end, the second packaging and expression vector includes a second ITR element or analogous element and a second expression cassette. The second expression cassette includes a polyA signal, a target gene, and a promoter of a negative strand.

The positive strand and negative strand undergo complementary base pairing.

In some embodiments, the positive strand and the negative strand undergo partial complementary base pairing.

In some embodiments, the first ITR element or analogous element and the second ITR element or analogous element are AAV viral ITR elements or analogous elements.

In some embodiments, the AAV viral ITR element includes a Rep binding element (RBE), a D region sequence, a Rep terminal resolution site (trs), an A sequence, a B sequence, and a C sequence.

In some embodiments, the analogous element is one or more of a DttRR element and a DtrsA′B′ element.

In some embodiments, the DttRR element is composed of a Rep binding element (RBE) and a Rep terminal resolution site (trs).

In some embodiments, the DtrsA′B′ element is composed of a D region sequence, a trs sequence, an A′ sequence, and a B′ sequence in an AAV ITR element.

In some embodiments, the ITR element has a nucleotide sequence of SEQ ID NO: 32.

In some embodiments, the DttRR element has a nucleotide sequence of SEQ ID NO: 33.

In some embodiments, the DtrsA′B′ element has a nucleotide sequence of SEQ ID NO: 34.

In some embodiments, the promoter is CMV, CAG, Ef1-α, or hU6. The promoter may also be selected from other gene expression promoters.

In some embodiments, the large gene is one or more of modified and expanded EGFP, modified and expanded luciferase, and modified and expanded CRISPR/CAS9. In some embodiments, the CRISPR/CAS9 is spCas9.

In some embodiments, the positive strand of the first packaging and expression vector and the negative strand of the second packaging and expression vector undergo partial complementary base pairing at 3′ ends to synthesize a second strand, thereby forming a complete expression cassette of the target gene.

The second aspect of the present disclosure provides a recombinant adeno-associated virus vector system, including a first recombinant adeno-associated virus vector and a second recombinant adeno-associated virus vector. The first recombinant adeno-associated virus vector includes the first packaging and expression vector as described above, and the second recombinant adeno-associated virus vector includes the second packaging and expression vector as described above.

In some embodiments, after co-transducing the host cell with the first recombinant adeno-associated virus vector and the second recombinant adeno-associated virus vector, the DNA molecules of the two recombinant adeno-associated virus vectors undergo partial complementary base pairing at 3′ end, initiating DNA synthesis to form a complete expression cassette for the large gene.

The third aspect of the present disclosure provides a host cell, wherein the host cell is transduced with the recombinant adeno-associated virus vector system as described above.

In some embodiments, the host cell is human-derived or from other mammals.

The fourth aspect of the present disclosure provides a use of the construction system as described above, or the recombinant adeno-associated virus vector system as described above, in the preparation of gene therapy drugs and/or vaccines, or for gene editing.

The fifth aspect of the present disclosure provides a gene therapy drug, wherein the gene therapy drug includes the construction system as described above or the recombinant adeno-associated virus vector system as described above.

The sixth aspect of the present disclosure provides a gene editing method, wherein the method includes using the construction system as described above or the recombinant adeno-associated virus vector system as described above.

The seventh aspect of the present disclosure provides a genetic recombination method, wherein the method includes using the construction system as described above or the recombinant adeno-associated virus vector system as described above.

Compared with prior art, the present disclosure provides the following advantageous effects:

The recombinant adeno-associated virus vector for large gene expression of the present application is capable of efficiently packaging the left and right fragments of the expression cassette of the large gene. The left and right fragments are respectively a positive strand and a negative strand, which are in a complementary relationship. Specifically, the left and right fragments undergo partial complementary base pairing at 3′ end to form a partially double-stranded structure. Subsequently, DNA synthesis extends the complementary strand, resulting in the formation of a complete large-gene expression cassette, which enables efficient expression of the large gene.

### BRIEF DESCRIPTION OF THE DRAWINGS

FIG. 1 shows a schematic diagram of the DNA sequence structure of AAV2 Flip ITR (RBE and trs sites are illustrated).

FIG. 2 shows a schematic diagram of the construction and packaging process of a recombinant adeno-associated virus vector of the present disclosure.

FIG. 3 shows a schematic diagram of the construction and packaging process of a lgAAV-CMV-Luci-7.6kb recombinant adeno-associated virus vector of the present disclosure.

FIG. 4 shows a schematic diagram of the construction and packaging process of a lgAAV-CMV-EGFP-8.3kb recombinant adeno-associated virus vector of the present disclosure.

FIG. 5 shows a schematic diagram of the construction and packaging process of a lgAAV-spCAS9-gRNA-NFkB-7.0kb recombinant adeno-associated virus vector of the present disclosure.

FIG. 6 shows an SDS-PAGE electrophoresis diagram of capsid proteins from the rAAV-EGFP recombinant adeno-associated virus vector, lgAAV-CMV-Luci-7.6kb recombinant adeno-associated virus vector, lgAAV-CMV-EGFP-8.3kb recombinant adeno-associated virus vector, and lgAAV-spCAS9-gRNA-NFkB-7.0kb recombinant adeno-associated virus vector of the present disclosure, wherein MW denotes the protein molecular weight standard, lane 1 denotes rAAV-EGFP recombinant adeno-associated virus vector, lane 2 denotes the lgAAV-CMV-Luci-7.6kb recombinant adeno-associated virus vector, lane 3 denotes the lgAAV-CMV-EGFP-8.3kb recombinant adeno-associated virus vector, and lane 4 denotes the lgAAV-spCAS9-gRNA-NFkB-7.0kb recombinant adeno-associated virus vector.

FIG. 7 shows a neutral agarose gel electrophoresis diagram of DNA from the rAAV-EGFP recombinant adeno-associated virus vector, lgAAV-CMV-Luci-7.6kb recombinant adeno-associated virus vector, lgAAV-CMV-EGFP-8.3kb recombinant adeno-associated virus vector, and lgAAV-spCAS9-gRNA-NFkB-7.0kb recombinant adeno-associated virus vector of the present disclosure, wherein MW denotes the DNA molecular weight standard, lane 1 denotes rAAV-EGFP recombinant adeno-associated virus vector, lane 2 denotes the lgAAV-CMV-Luci-7.6kb recombinant adeno-associated virus vector, lane 3 denotes the lgAAV-CMV-EGFP-8.3kb recombinant adeno-associated virus vector, and lane 4 denotes the lgAAV-spCAS9-gRNA-NFkB-7.0kb recombinant adeno-associated virus vector.

FIG. 8 shows an alkaline agarose gel electrophoresis diagram of DNA from the rAAV-EGFP recombinant adeno-associated virus vector, lgAAV-CMV-Luci-7.6kb recombinant adeno-associated virus vector, lgAAV-CMV-EGFP-8.3kb recombinant adeno-associated virus vector, and lgAAV-spCAS9-gRNA-NFkB-7.0kb recombinant adeno-associated virus vector of the present disclosure, wherein MW denotes the DNA molecular weight standard, lane 1 denotes rAAV-EGFP recombinant adeno-associated virus vector, lane 2 denotes the lgAAV-CMV-Luci-7.6kb recombinant adeno-associated virus vector, lane 3 denotes the lgAAV-CMV-EGFP-8.3kb recombinant adeno-associated virus vector, and lane 4 denotes the lgAAV-spCAS9-gRNA-NFkB-7.0kb recombinant adeno-associated virus vector.

FIG. 9 shows fluorescence detection images of EGFP expression in mouse eye retina following injection with the lgAAV-CMV-EGFP-8.3kb recombinant adeno-associated virus vector of the present disclosure.

FIG. 10 shows fluorescence detection images of Luciferase expression in mouse muscle following injection with the lgAAV-CMV-Luci-7.6kb recombinant adeno-associated virus vector of the present disclosure.

FIG. 11 shows functional validation of large gene expression in HEK293 cells transduced with the lgAAV-spCAS9-gRNA-NFkB-7.0kb recombinant adeno-associated virus vector of the present disclosure.

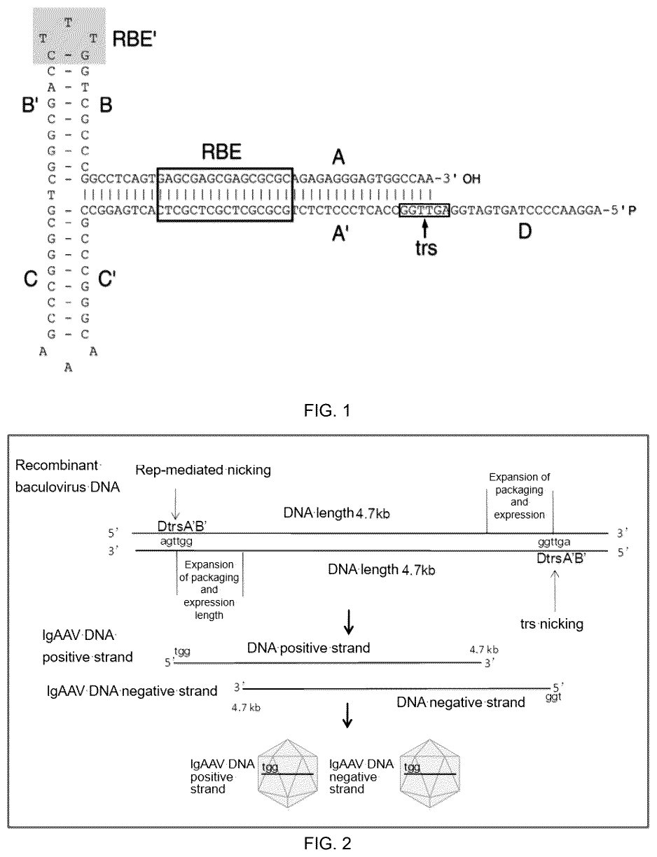

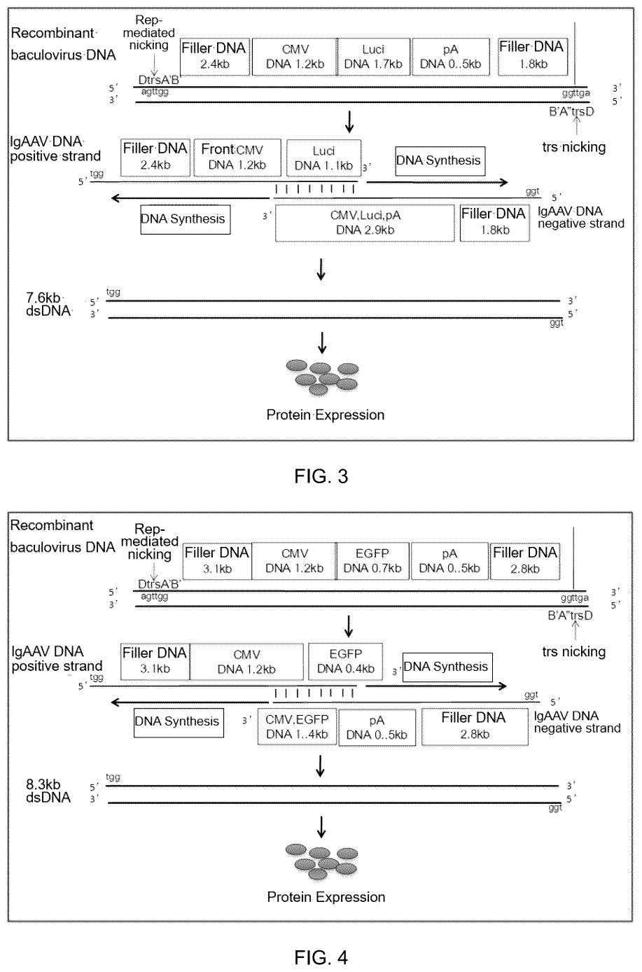

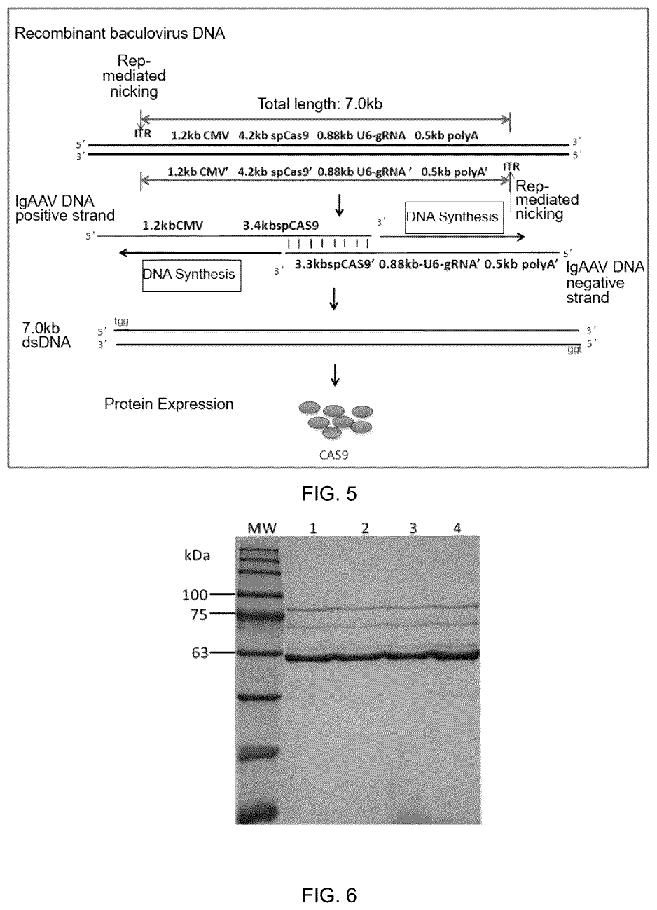

### DETAILED DESCRIPTION

The present disclosure provides a viral vector based the recombinant adeno-associated virus (AAV), wherein this viral vector can package and express large genes.

The present disclosure has developed a design and packaging technology for a recombinant adeno-associated virus vector capable of packaging and expressing large genes (referred to as lgAAV vector below). The primary principle of the packaging and production of lgAAV vectors lies in the use of two complementary single-stranded, single-polarity AAV DNA molecules, which undergo complementary base pairing via homologous sequences within host cells, then DNA synthesis is initiated from 3′ end to form complete double-stranded DNA molecules, thereby enabling protein expression. During the packaging of single-stranded, single-polarity DNA into AAV vectors, ITR sequences (i.e., DtrsA′B′BC′CA), modified ITR sequences (which are analogous elements that contain only the Rep binding element (RBE) and Rep terminal resolution site (trs)), or DtrsA′B′ are employed. The above mentioned ITR, RBE-trs, or DtrsA′B′ packaging element is positioned at the 5′ ends of the positive strand and negative strand of the large gene to be packaged. As a result, the packaging and production of positive strand vectors and negative strand vectors based on the recombinant adeno-associated virus for large gene expression are realized. In application, equal amounts of the positive strand vectors and negative strand vectors are mixed and used to transduce host cells, leading to expression of the large gene. The packaged positive strand and negative strand DNA molecules in rAAV exhibit partial complementarity at 3′ end. They undergo complementary base pairing within host cells and DNA synthesis is then initiated from 3′ end, forming double-stranded DNA molecules of the desired length which is subsequently transcribed to express the target protein. The packaging method of the lgAAV vector of the present disclosure differs from the existing rAAV based large-gene expression methods. Conventional rAAV vector based large-gene expression methods rely on homologous recombination between double-stranded DNA molecules and RNA splicing, resulting in low efficiency, poor performance, and limited applicability. In contrast, the lgAAV vector based method for large gene packaging and expression of the present disclosure relies on two separately packaged single-stranded, single-polarity DNA molecules in AAV vectors. Complementary base pairing between these two single-stranded DNA molecules, followed by DNA synthesis at 3′ end, leads to the formation of a double-stranded DNA structure, which enables transcription and expression of the protein. This approach achieves high efficiency, accuracy, and stability. Therefore, this approach can be effectively applied to the expression of proteins encoded by large genes and AAV-mediated gene therapy of diseases caused by large genes. The present disclosure provides a recombinant adeno-associated virus (lgAAV) vector for large gene expression, which may include a conventional or modified AAV protein capsid, and variant AAV genomic components.

Adeno-associated virus (AAV) is a single-stranded DNA virus commonly used as a vector in gene therapy to deliver exogenous genes into patients. The AAV genome is a linear, single-stranded DNA (ssDNA) of approximately 4.7 kb in length. It features 145 bp inverted terminal repeats (ITRs) at both ends and two open reading frames (ORFs) in the middle. Each ITR includes a 125-nucleotide palindromic sequence serving as the initiation element for replication, and this sequence can achieve self-folding to form a hairpin structure. The ITR is the only cis-regulatory element in the AAV life cycle. One ORF encodes replication proteins (Rep), while the other ORF encodes capsid proteins (Cap). AAV is a naturally replication-defective virus and requires helper viruses, such as adenovirus (AV) or herpes simplex virus (HSV-1), to complete its replication cycle. In the absence of helper viruses, the AAV genome integrates into the host genome and persists latently as a provirus.

The ITR plays a critical role in viral replication and packaging, and participates in viral genome integration and excision (rescue) within the host genome. The ITR sequence has a CG content exceeding 80%. The first 125 bp (positions 1-125) of the ITR sequence can be sequentially divided into distinct segments designated as A, B, B′, C′, C, A′, etc., wherein A and A′, B and B′, C and C′ are respectively complementary and form a T-shaped hairpin structure serving as the origin for AAV DNA self-replication. The subsequent 20 bp following the first 125 bp of the ITR sequence form a unique D sequence.

### A-A′ Region

The tandem repeat sequence 5′-GAGCGAGC GAGC GCGC-3‘ within the A-A’ region is the core sequence of both the A-A′ region and the entire ITR. Its most clearly defined function is binding to the Rep protein, hence it is referred to as the Rep binding element (RBE). RBE is involved in replication, transcription, and proviral integration of the AAV genome. Nearly all crucial ITR functions are initiated by interaction between Rep and RBE. Within the A-A′ region, a specific terminal resolution site (trs) sequence is formed. The trs sequence within ITR is involved in AAV rescue and replication pathways.

B-B′ and C-C′ regions: these two palindromic structures also contribute to Rep binding to some extent. The sequence designated as RBE′ (5′-CTTTG-3′), located in the B-B′ region, exhibits strong affinity for Rep.

The A-A′, B-B′, and C-C′ regions within ITR all form palindromic double-stranded structures, while only the D region remains single-stranded.

In the present disclosure, the first packaging and expression vector, positive strand vector, and positive vector are interchangeable terms. The second packaging and expression vector, negative strand vector, and negative vector are interchangeable terms.

The first aspect of the present disclosure provides a construction system of a recombinant adeno-associated virus vector for large gene packaging and expression, wherein a to-be-packaged DNA may have a length exceeding 4.7 kb. The construction system includes a first packaging and expression vector and a second packaging and expression vector.

From 5′ end to 3′ end, the first packaging and expression vector includes a first ITR element or analogous element and a first expression cassette. The first expression cassette includes a promoter, a target gene, and a polyadenylation (polyA) signal of a positive strand.

From 5′ end to 3′ end, the second packaging and expression vector includes a second ITR element or analogous element and a second gene expression cassette. The second expression cassette includes a polyA signal, a target gene, and a promoter of a negative strand.

The positive strand and the negative strand undergo complementary base pairing.

In the present disclosure, the first packaging and expression vector is a single-stranded DNA, which includes, from 5′ end to 3′ end, an ITR element or analogous element, a promoter, a target gene, and a polyA signal. The second packaging and expression vector is a single-stranded DNA, which includes, from 5′ end to 3′ end, a complementary strand of the ITR element or analogous element, and a target gene. The base pairing between the positive strand and the negative strand is partially complementary, and the length of the complementary region is no less than 1 kb (e.g., 1.1 kb, 1.8 kb, or 2.4 kb). The principle of the present disclosure involves that the single-stranded, single-polarity DNA packaged by AAV undergoes partial complementary base pairing within host cells, which subsequently serves as a template for DNA synthesis to generate a complete expression cassette of the large gene, thereby enabling expression of corresponding proteins encoded by the large gene.

In the construction system of the present disclosure, the ITR element or analogous element is one or more of an AAV viral ITR element, a DttRR element, and a DtrsA′B′ element. In some embodiments, the AAV viral ITR element includes a Rep binding element (RBE), a D region sequence, a Rep terminal resolution site (trs), an A sequence, a B sequence, and a C sequence. In some embodiments, the ITR element has a nucleotide sequence of SEQ ID NO: 32. In some embodiments, the DttRR element is composed of a Rep binding element (RBE) and a Rep terminal resolution site (trs). In some embodiments, the DttRR element has a nucleotide sequence of SEQ ID NO: 33. In some embodiments, the DtrsA′B′ element is composed of a D region sequence, a trs sequence, an A′ sequence, and a B′ sequence in an AAV ITR element. In some embodiments, the DtrsA′B′ element has a nucleotide sequence of SEQ ID NO: 34.

ITR Sequence:
(SEQ ID NO: 32)
aggaacccctagtgatggagttggccactccctctctgcgcgctcgctc
gctcactgaggccgggcgaccaaaggtcgcccgacgcccgggctttgcc
cgggcggcctcagtgagcgagcgagcgcgcagagagggagtggccaa
DttRR Sequence:
(SEQ ID NO: 33)
aggaacccctagtgatggagttggagttggctgcgcgctcgctcgctca
ctgctgcgcgctcgctcgctcactg
DtrsA′B′ Sequence:
(SEQ ID NO: 34)
aggaacccctagtgatggagttggccactccctctctgcgcgctcgctc
gctcactgaggccgggcgaccaaa

In the construction system of the present disclosure, the 3′ end of the positive strand expression vector does not include an ITR element or analogous element, and the 3′ end of the negative strand expression vector does not include a complementary strand of the ITR element or analogous element.

In the construction system of the present disclosure, the large gene is a gene having a length exceeding 4.7 kb.

In the construction system of the present disclosure, a linker sequence is provided between the ITR element or analogous element and the promoter in the positive strand expression vector. A linker sequence is provided between the complementary strand of the ITR element or analogous element and a second fragment of the target gene in the negative strand expression vector. In some specific embodiments, the linker sequence is stuff1 or stuff2.

In the construction system of the present disclosure, the complementary region between a first fragment of the target gene and a second fragment of the target gene has a length of no less than 1 kb.

In the construction system of the present disclosure, the promoter is one of CMV, CAG, Ef1-α, and hU6. The promoter may also be selected from other gene expression promoters.

In the construction system of the present disclosure, the positive strand of the first packaging and expression vector and the negative strand of the second packaging and expression vector undergo partial complementary base pairing at 3′ end, followed by a second strand synthesis, thereby forming a complete expression cassette of the target gene.

In the construction system of the present disclosure, the large gene is one or more of modified and expanded EGFP, modified and expanded luciferase, and modified and expanded CRISPR/CAS9. In some embodiments, the CRISPR/CAS9 is spCas9. In one specific embodiment, using the modified and expanded EGFP as an example, the construction system DttRR-3.1kbstuff1-CEP-2.8kbstuff2-R′R′t′t′D′ has a nucleotide sequence of SEQ ID NO: 29. Taking the modified and expanded luciferase as an example, the construction system DttRR-2.4kb stuff1-CLP-1.8kb stuff2-R′R′t′t′D′ has a nucleotide sequence of SEQ ID NO: 30. Taking the modified and expanded CRISPR/CAS9 as an example, the construction system ITRL-spCas9-gRNA12(NFkB) has a nucleotide sequence of SEQ ID NO: 31.

DttRR-3.1kbstuff1-CEP-2.8kbstuff2-R′R′t′t′D′:
(SEQ ID NO: 29)
aggaacccctagtgatggagttggagttggctgcgcgctcgctcgctcactgctgcgcgctcgctcgctcactgacgc
gtgaagaaagacggcgaagtgcggggcagcatcaacagattcaagaccagcgactacgtgaaagaagccaaacagctgctga
aggtgcagaaggcctaccaccagctggaccagagcttcatcgacacctacatcgacctgctggaaacccggcggacctactatg
agggacctggcgagggcagccccttcggctggaaggacatcaaagaatggtacgagatgctgatgggccactgcacctacttc
cccgaggaactgcggagcgtgaagtacgcctacaacgccgacctgtacaacgccctgaacgacctgaacaatctcgtgatcacc
agggacgagaacgagaagctggaatattacgagaagttccagatcatcgagaacgtgttcaagcagaagaagaagcccaccct
gaagcagatcgccaaagaaatcctcgtgaacgaagaggatattaagggctacagagtgaccagcaccggcaagcccgagttca
ccaacctgaaggtgtaccacgacatcaaggacattaccgcccggaaagagattattgagaacgccgagctgctggatcagattg
ccaagatcctgaccatctaccagagcagcgaggacatccaggaagaactgaccaatctgaactccgagctgacccaggaagag
atcgagcagatctctaatctgaagggctataccggcacccacaacctgagcctgaaggccatcaacctgatcctggacgagctgt
ggcacaccaacgacaaccagatcgctatcttcaaccggctgaagctggtgcccaagaaggtggacctgtcccagcagaaagag
atccccaccaccctggtggacgacttcatcctgagccccgtcgtgaagagaagcttcatccagagcatcaaagtgatcaacgcca
tcatcaagaagtacggcctgcccaacgacatcattatcgagctggcccgcgagaagaactccaaggacgcccagaaaatgatca
acgagatgcagaagcggaaccggcagaccaacgagcggatcgaggaaatcatccggaccaccggcaaagagaacgccaag
tacctgatcgagaagatcaagctgcacgacatgcaggaaggcaagtgcctgtacagcctggaagccatccctctggaagatctg
ctgaacaaccccttcaactatgaggtggaccacatcatccccagaagcgtgtccttcgacaacagcttcaacaacaaggtgctcgt
gaagcaggaagaaaacagcaagaagggcaaccggaccccattccagtacctgagcagcagcgacagcaagatcagctacga
aaccttcaagaagcacatcctgaatctggccaagggcaagggcagaatcagcaagaccaagaaagagtatctgctggaagaac
gggacatcaacaggttctccgtgcagaaagacttcatcaaccggaacctggtggataccagatacgccaccagaggcctgatga
acctgctgcggagctacttcagagtgaacaacctggacgtgaaagtgaagtccatcaatggcggcttcaccagctttctgcggcg
gaagtggaagtttaagaaagagcggaacaaggggtacaagcactgacagccctgctgctgcctctggctctgctgctgcatgcc
gctagacccgattacaaggatgacgacgataagggtggtggtggttctatccagatgacacagactacatcctccctgtctgcctct
ctgggagacagagtcaccatcagttgcagggcaagtcaggacattagcaattttttaaactggtatcagcagaaaccagatggaac
tgttaaactcctgatctacgcgacatcaagattacattctggagtcccatcaaggttcagtggcagtgggtctggaacagatttttctct
caccattagcaaactggagcaagaagatattgccacttacttttgccaacagggtaatacgcttccacttacgttcggcgctgggac
aaagttggaacttaaaggtggtggtggttctggcggcggcggctccggaggaggaggatcgctgcaacagtctggacctgagtt
ggtgaagcctggggcttcagtgaagatttcctgcaaggcttctggatacacattcactgactactacatgaattgggtgaagcttagc
catggaaagagccttgagtggattggagatattgttcctaacaatggtgatactacttacaaccagaatttcagaggcaaggccaca
ttgactgtagacaagtcctccagcacagcctacatggagctccgcagcctgacatctgaggactctgcagtctattactgtgcaaga
ttcagtaattacgtttacccttttgactactggggccaaggcaccactatcacagtctccaccacgacgccagcgccgcgaccacc
aacaccggcgcccaccatcgcgtcgcagcccctgtccctgcgcccagaggcgtgccggccagcggggggggcgcagtgca
cacgagggggctggacttcgcctgtgatatctacatctgggcgcccttggccgggacttgtggggtccttctcctgtcactggttat
caccctttactgcaaacggggcagaaagaaactcctgtatatattcaaacaaccatttatgagaccagtacaaactactcaagagga
agatggctgtagctgccgatttccagaagaagaagaaggaggatgtgaactgagagtgaagttcagcaggagcgcagacgccc
ccgcgtaccagcagggccagaaccagctctataacgagctcaatctaggacgaagagaggagtacgatgttttggacaagaga
cgtggccgggaccctgagatggggggaaagccgagaaggaagaaccctcaggaaggcctgtacaatgaactgcagaaagat
aagatggcggaggcctacagtgagattgggatgaaaggcgagcgccggaggggcaaggggcacgatggcctttaccagggt
ctcagtacagccaccaaggacacctacgacgcccttcacatgcaggccctgccccctcgctaaggatacgcgtctagttattaata
gtaatcaattacggggtcattagttcatagcccatatatggagttccgcgttacataacttacggtaaatggcccgcctggctgaccg
cccaacgacccccgcccattgacgtcaataatgacgtatgttcccatagtaacgtcaatagggactttccattgacgtcaatgggtg
gagtatttacggtaaactgcccacttggcagtacatcaagtgtatcatatgccaagtacgccccctattgacgtcaatgacggtaaat
ggcccgcctggcattatgcccagtacatgaccttatgggactttcctacttggcagtacatctacgtattagtcatcgctattaccatg
gtgatgcggttttggcagtacatcaatgggcgtggatagcggtttgactcacggggatttccaagtctccaccccattgacgtcaat
gggagtttgttttgcaccaaaatcaacgggactttccaaaatgtcgtaacaactccgccccattgacgcaaatgggcggtaggcgt
gtacggtgggaggtctatataagcagagctcgtttagtgaaccgtcagatcgcctggagacgccatccacgctgttttgacctccat
agaagacaccgggaccgatccagcctccgcggattcgaatcccggccgggaacggtgcattggaacgcggattccccgtgcc
aagagtgacgtaagtaccgcctatagagtctataggcccacaaaaaatgctttcttcttttaatatacttttttgtttatcttatttctaatac
tttccctaatctctttctttcagggcaataatgatacaatgtatcatgcctctttgcaccattctaaagaataacagtgataatttctgggtt
aaggcaatagcaatatttctgcatataaatatttctgcatataaattgtaactgatgtaagaggtttcatattgctaatagcagctacaat
ccagctaccattctgcttttattttatggttgggataaggctggattattctgagtccaagctaggcccttttgctaatcatgttcatacct
cttatcttcctcccacagctcctgggcaacgtgctggtctgtgtgctggcccatcactttggcaaagaattgggattcgaacatcgatt
gaattccccggggatccatggtgagcaagggcgaggagctgttcaccggggtggtgcccatcctggtcgagctggacggcgac
gtaaacggccacaagttcagcgtgtccggcgagggcgagggcgatgccacctacggcaagctgaccctgaagttcatctgcac
caccggcaagctgcccgtgccctggcccaccctcgtgaccaccctgacctacggcgtgcagtgcttcagccgctaccccgacca
catgaagcagcacgacttcttcaagtccgccatgcccgaaggctacgtccaggagcgcaccatcttcttcaaggacgacggcaa
ctacaagacccgcgccgaggtgaagttcgagggcgacaccctggtgaaccgcatcgagctgaagggcatcgacttcaaggag
gacggcaacatcctggggcacaagctggagtacaactacaacagccacaacgtctatatcatggccgacaagcagaagaacgg
catcaaggtgaacttcaagatccgccacaacatcgaggacggcagcgtgcagctcgccgaccactaccagcagaacaccccca
tcggcgacggccccgtgctgctgcccgacaaccactacctgagcacccagtccgccctgagcaaagaccccaacgagaagcg
cgatcacatggtcctgctggagttcgtgaccgccgccgggatcactctcggcatggacgagctgtacaagtaactctcgagagat
ctacgggtggcatccctgtgacccctccccagtgcctctcctggccctggaagttgccactccagtgcccaccagccttgtcctaat
aaaattaagttgcatcattttgtctgactaggtgtccttctataatattatggggtggaggggggtggtatggagcaaggggcaagtt
gggaagacaacctgtagggcctgcggggtctattgggaaccaagctggagtgcagtggcacaatcttggctcactgcaatctcc
gcctcctgggttcaagcgattctcctgcctcagcctcccgagttgttgggattccaggcatgcatgaccaggctcagctaatttttgtt
tttttggtagagacggggtttcaccatattggccaggctggtctccaactcctaatctcaggtgatctacccaccttggcctcccaaat
tgctgggattacaggcgtgaaccactgctcccttccctgtccttctgattttgtaggtaaccaaccgtaatccggacctcacccagct
ccctggtggctttcatcaactctcgctgtacatctccgggcggttcctacggccatctctccattggtaccatgagcccttctttaggat
tcccacctcagatgagtcatcaaaaaggaacttcacctccctatggagtccagccctgtgtaccacatgactctactcggggttcaa
tgatgcttcacccccagtcccggggaccacgtgcaacctgccagctgaagtcagagctggatatgatggttggcaagtgcccgg
aggaccctttggaaggggacatgtctagccccaactccacaggcacacaggatcacctgttggggatgctggatgggcgggag
gacctggagagagaggagaagcctgagcctgagtctgtgtatgagacagactgccgctgggatggttgcagccaggagttcgat
tcccaggagcagctggtgcaccacatcaacagtgagcatatccacggggagcggaaggaattcgtgtgccattggggaggttgc
tccagggagctgaggcccttcaaggcccaatacatgctggtggtgcacatgcgcagacacacgggcgagaagccacacaagt
gcacgtttgaaggctgtcggaagtcctattcacgccttgaaaacctcaagacgcaccttcggtcgcacacgggtgagaagccttac
atgtgtgagcaagaaggttgcagcaaggcctttagcaatgccagtgaccgcgccaagcaccagaatcggacccactccaatgag
aagccatacgtgtgcaagctccccggctgcaccaagcgctacacagatcccagctcgctccgcaaacacgtgaagacagtgcat
ggtccggatgcccacgtgaccaagcggcatcgaggggatggccccttgccacgggctcagcccctctccacagtggagccca
agcgggaaagggaaggaggatccggcagggaagagagcagactgactgtgcccgagagtgccatgccgcagcagagcccc
ggagcgcagtcctcttgcagcagcgaccactccccagcaggcagtgcggccaacacggacagcggcgtggagatggccggc
aacgccgggggcagcactgaggacttgtccagcttggatgaaggaccttgtgtctcggccaccggactctccacgcttcgccgc
ctggagaaccttaggctggatcagctgcatcagctccggcccatagggtctcggggtctcaaactgcccagcttaacccacgctg
gcgcacctgtgtctcgccgtctgggccccccagtctccctggaccgccgcagcagcagctccagcagcatgagctctgcttaca
cagtcagccgcaggtcctccctggcatcccctttcccgccgggaaccccaccagagaatggggcatcgtcactacctggcctca
cacctgctcagcactacatgctccgtgccagatatgcttcagccagggggagtggcaccccgcccactgcagctcacagcctgg
atcggatgggaggtctttctgttcctccttggagaagccgaaccgagtacccgggatacaacccaaatgcaggggtcactcggag
ggccagtgacccagcccgggctgctgaccacccagctccagccagagtccagcggttcaagagcctgggatgtgtccacacgc
cccctagtgtggcaacgggacggaacttcgatccccaccaccctacctctgtctattcgccacagccccccagcatcaccgaaaa
tgttgccatggatactagggggctacaggaggagccagaggttggaacttctgtgatgggcaatggtctgaacccatacatggatt
tttcctccactgatactctgggatatgggggacccgaggggacggcagctgagccttatgaagctaggggtccaggttccctgcct
cttgggcctggtccaccaaccaactatggccctggccactgtgcccagcaggtctcctatcctgatcccaccccagaaaactggg
gtgagttcccttctcatgctggggtgtaccctagcaataaggctccgggtgctgcctatagccagtgtcctcgacttgagcattatgg
acaagtgcaggtaaaaccagaacaagggtgcccagtggggtctgactccaccggattggcaccctgcctcaatgcccacccca
gtgaagggtccccaggcccgcagcctctgttttcacatcatccccagctccctcagccccagtatccccagtcgggtccctatcctc
agcctccccatggttatctctcaacagaacccaggcttggcctcaatttcaacccctcctcctctcattccacaggacagctcaaagc
tcagctggtgtgtaattacgttcagtcgcagcaggaattgttgtgggagggaagaaaccggggagggctccccaaccaggaact
cccataccagagccccaagtttctggggggttcccaagttagtcagagccctgccaagaccccagcagcagcggcggcagcat
atggatctggctttgcacctgcttcggccaatcacaaatcaggctcctatcctgccccttcaccctgccatgaaactttcaccgtggg
agtaaacaggccttcccacaggccagcagcaccaccccgacttctgcccccgctgtccccttgctatgggcccctcaaggtggg
ggataccaaccccagctgtggccatcctgaggtgggcaggttaggagcaggccctgccttgtaccctggtaaccacgtg
DttRR-2.4kb stuff1-CLP-1.8kb stuff2-R′R′t′t′D′ :
(SEQ ID NO: 30)
aggaacccctagtgatggagttggagttggctgcgcgctcgctcgctcactgctgcgcgctcgctcgctcactgacgc
gtggctgttcaaagaggccaacgtggaaaacaacgagggcaggcggagcaagagaggcgccagaaggctgaagcggcgga
ggcggcatagaatccagagagtgaagaagctgctgttcgactacaacctgctgaccgaccacagcgagctgagcggcatcaac
ccctacgaggccagagtgaagggcctgagccagaagctgagcgaggaagagttctctgccgccctgctgcacctggccaaga
gaagaggcgtgcacaacgtgaacgaggtggaagaggacaccggcaacgagctgtccaccaaagagcagatcagccggaac
agcaaggccctggaagagaaatacgtggccgaactgcagctggaacggctgaagaaagacggcgaagtgcggggcagcatc
aacagattcaagaccagcgactacgtgaaagaagccaaacagctgctgaaggtgcagaaggcctaccaccagctggaccaga
gcttcatcgacacctacatcgacctgctggaaacccggcggacctactatgagggacctggcgagggcagccccttcggctgga
aggacatcaaagaatggtacgagatgctgatgggccactgcacctacttccccgaggaactgcggagcgtgaagtacgcctaca
acgccgacctgtacaacgccctgaacgacctgaacaatctcgtgatcaccagggacgagaacgagaagctggaatattacgag
aagttccagatcatcgagaacgtgttcaagcagaagaagaagcccaccctgaagcagatcgccaaagaaatcctcgtgaacgaa
gaggatattaagggctacagagtgaccagcaccggcaagcccgagttcaccaacctgaaggtgtaccacgacatcaaggacatt
accgcccggaaagagattattgagaacgccgagctgctggatcagattgccaagatcctgaccatctaccagagcagcgaggac
atccaggaagaactgaccaatctgaactccgagctgacccaggaagagatcgagcagatctctaatctgaagggctataccggc
acccacaacctgagcctgaaggccatcaacctgatcctggacgagctgtggcacaccaacgacaaccagatcgctatcttcaac
cggctgaagctggtgcccaagaaggtggacctgtcccagcagaaagagatccccaccaccctggtggacgacttcatcctgag
ccccgtcgtgaagagaagcttcatccagagcatcaaagtgatcaacgccatcatcaagaagtacggcctgcccaacgacatcatt
atcgagctggcccgcgagaagaactccaaggacgcccagaaaatgatcaacgagatgcagaagcggaaccggcagaccaac
gagcggatcgaggaaatcatccggaccaccggcaaagagaacgccaagtacctgatcgagaagatcaagctgcacgacatgc
aggaaggcaagtgcctgtacagcctggaagccatccctctggaagatctgctgaacaaccccttcaactatgaggtggaccacat
catccccagaagcgtgtccttcgacaacagcttcaacaacaaggtgctcgtgaagcaggaagaaaacagcaagaagggcaacc
ggaccccattccagtacctgagcagcagcgacagcaagatcagctacgaaaccttcaagaagcacatcctgaatctggccaagg
gcaagggcagaatcagcaagaccaagaaagagtatctgctggaagaacgggacatcaacaggttctccgtgcagaaagacttc
atcaaccggaacctggtggataccagatacgccaccagaggcctgatgaacctgctgcggagctacttcagagtgaacaacctg
gacgtgaaagtgaagtccatcaatggcggcttcaccagctttctgcggcggaagtggaagtttaagaaagagcggaacaagggg
tacaagcactgacagccctgctgctgcctctggctctgctgctgcatgccgctagacccgattacaaggatgacgacgataaggg
tggtggtggttctatccagatgacacagactacatcctccctgtctgcctctctgggagacagagtcaccatcagttgcagggcaag
tcaggacattagcaattttttaaactggtatcagcagaaaccagatggaactgttaaactcctgatctacgcgacatcaagattacatt
ctggagtcccatcaaggttcagtggcagtgggtctggaacagatttttctctcaccattagcaaactggagcaagaagatattgcca
cttacttttgccaacagggtaatacgcttccacttacgttcggcgctgggacaaagttggaacgcgtctagttattaatagtaatcaatt
acggggtcattagttcatagcccatatatggagttccgcgttacataacttacggtaaatggcccgcctggctgaccgcccaacga
cccccgcccattgacgtcaataatgacgtatgttcccatagtaacgtcaatagggactttccattgacgtcaatgggtggagtatttac
ggtaaactgcccacttggcagtacatcaagtgtatcatatgccaagtacgccccctattgacgtcaatgacggtaaatggcccgcct
ggcattatgcccagtacatgaccttatgggactttcctacttggcagtacatctacgtattagtcatcgctattaccatggtgatgcggt
tttggcagtacatcaatgggcgtggatagcggtttgactcacggggatttccaagtctccaccccattgacgtcaatgggagtttgttt
tgcaccaaaatcaacgggactttccaaaatgtcgtaacaactccgccccattgacgcaaatgggcggtaggcgtgtacggtggga
ggtctatataagcagagctcgtttagtgaaccgtcagatcgcctggagacgccatccacgctgttttgacctccatagaagacacc
gggaccgatccagcctccgcggattcgaatcccggccgggaacggtgcattggaacgcggattccccgtgccaagagtgacgt
aagtaccgcctatagagtctataggcccacaaaaaatgctttcttcttttaatatacttttttgtttatcttatttctaatactttccctaatctc
tttctttcagggcaataatgatacaatgtatcatgcctctttgcaccattctaaagaataacagtgataatttctgggttaaggcaatagc
aatatttctgcatataaatatttctgcatataaattgtaactgatgtaagaggtttcatattgctaatagcagctacaatccagctaccatt
ctgcttttattttatggttgggataaggctggattattctgagtccaagctaggcccttttgctaatcatgttcatacctcttatcttcctccc
acagctcctgggcaacgtgctggtctgtgtgctggcccatcactttggcaaagaattgggattcgaacatcgatatggaagatgcc
aaaaacattaagaagggcccagcgccattctacccactcgaagacgggaccgccggcgagcagctgcacaaagccatgaagc
gctacgccctggtgcccggcaccatcgcctttaccgacgcacatatcgaggtggacattacctacgccgagtacttcgagatgag
cgttcggctggcagaagctatgaagcgctatgggctgaatacaaaccatcggatcgtggtgtgcagcgagaatagcttgcagttct
tcatgcccgtgttgggtgccctgttcatcggtgtggctgtggccccagctaacgacatctacaacgagcgcgagctgctgaacag
catgggcatcagccagcccaccgtcgtattcgtgagcaagaaagggctgcaaaagatcctcaacgtgcaaaagaagctaccgat
catacaaaagatcatcatcatggatagcaagaccgactaccagggcttccaaagcatgtacaccttcgtgacttcccatttgccacc
cggcttcaacgagtacgacttcgtgcccgagagcttcgaccgggacaaaaccatcgccctgatcatgaacagtagtggcagtac
cggattgcccaagggcgtagccctaccgcaccgcaccgcttgtgtccgattcagtcatgcccgcgaccccatcttcggcaaccag
atcatccccgacaccgctatcctcagcgtggtgccatttcaccacggcttcggcatgttcaccacgctgggctacttgatctgcggc
tttcgggtcgtgctcatgtaccgcttcgaggaggagctattcttgcgcagcttgcaagactataagattcaatctgccctgctggtgc
ccacactatttagcttcttcgctaagagcactctcatcgacaagtacgacctaagcaacttgcacgagatcgccagcggcggggc
gccgctcagcaaggaggtaggtgaggccgtggccaaacgcttccacctaccaggcatccgccagggctacggcctgacagaa
acaaccagcgccattctgatcacccccgaaggggacgacaagcctggcgcagtaggcaaggtggtgcccttcttcgaggctaa
ggtggtggacttggacaccggtaagacactgggtgtgaaccagcgcggcgagctgtgcgtccgtggccccatgatcatgagcg
gctacgttaacaaccccgaggctacaaacgctctcatcgacaaggacggctggctgcacagcggcgacatcgcctactgggac
gaggacgagcacttcttcatcgtggaccggctgaagagcctgatcaaatacaagggctaccaggtagccccagccgaactgga
gagcatcctgctgcaacaccccaacatcttcgacgccggggtcgccggcctgcccgacgacgatgccggcgagctgcccgcc
gcagtcgtcgtgctggaacacggtaaaaccatgaccgagaaggagatcgtggactatgtggccagccaggttacaaccgccaa
gaagctgcgcggtggtgttgtgttcgtggacgaggtgcctaaaggactgaccggcaagttggacgcccgcaagatccgcgagat
tctcattaaggccaagaagggcggcaagatcgccgtgtaactcgagagatctacgggtggcatccctgtgacccctccccagtgc
ctctcctggccctggaagttgccactccagtgcccaccagccttgtcctaataaaattaagttgcatcattttgtctgactaggtgtcct
tctataatattatggggtggaggggggtggtatggagcaaggggcaagttgggaagacaacctgtagggcctgcggggtctatt
gggaaccaagctggagtgcagtggcacaatcttggctcactgcaatctccgcctcctgggttcaagcgattctcctgcctcagcct
cccgagttgttgggattccaggcatgcatgaccaggctcagctaatttttgtttttttggtagagacggggtttcaccatattggccag
gctggtctccaactcctaatctcaggtgatctacccaccttggcctcccaaattgctgggattacaggcgtgaaccactgctcccttc
cctgtccttctgattttgtaggtaacctgccagctgaagtcagagctggatatgatggttggcaagtgcccggaggaccctttggaa
ggggacatgtctagccccaactccacaggcacacaggatcacctgttggggatgctggatgggcgggaggacctggagagag
aggagaagcctgagcctgagtctgtgtatgagacagactgccgctgggatggttgcagccaggagttcgattcccaggagcagc
tggtgcaccacatcaacagtgagcatatccacggggagcggaaggaattcgtgtgccattggggaggttgctccagggagctga
ggcccttcaaggcccaatacatgctggtggtgcacatgcgcagacacacgggcgagaagccacacaagtgcacgtttgaaggc
tgtcggaagtcctattcacgccttgaaaacctcaagacgcaccttcggtcgcacacgggtgagaagccttacatgtgtgagcaaga
aggttgcagcaaggcctttagcaatgccagtgaccgcgccaagcaccagaatcggacccactccaatgagaagccatacgtgtg
caagctccccggctgcaccaagcgctacacagatcccagctcgctccgcaaacacgtgaagacagtgcatggtccggatgccc
acgtgaccaagcggcatcgaggggatggccccttgccacgggctcagcccctctccacagtggagcccaagcgggaaaggg
aaggaggatccggcagggaagagagcagactgactgtgcccgagagtgccatgccgcagcagagccccggagcgcagtcct
cttgcagcagcgaccactccccagcaggcagtgcggccaacacggacagcggcgtggagatggccggcaacgccgggggc
agcactgaggacttgtccagcttggatgaaggaccttgtgtctcggccaccggactctccacgcttcgccgcctggagaaccttag
gctggatcagctgcatcagctccggcccatagggtctcggggtctcaaactgcccagcttaacccacgctggcgcacctgtgtct
cgccgtctgggccccccagtctccctggaccgccgcagcagcagctccagcagcatgagctctgcttacacagtcagccgcag
gtcctccctggcatcccctttcccgccgggaaccccaccagagaatggggcatcgtcactacctggcctcacacctgctcagcac
tacatgctccgtgccagatatgcttcagccagggggagtggcaccccgcccactgcagctcacagcctggatcggatgggaggt
ctttctgttcctccttggagaagccgaaccgagtacccgggatacaacccaaatgcaggggtcactcggagggccagtgaccca
gcccgggctgctgaccacccagctccagccagagtccagcggttcaagagcctgggatgtgtccacacgccccctagtgtggc
aacgggacggaacttcgatccccaccaccctacctctgtctattcgccacagccccccagcatcaccgaaaatgttgccatggata
ctagggggctacaggaggagccagaggttggaacttctgtgatgggcaatggtctgaacccatacatggatttttcctccactgata
ctctgggatatgggggacccgaggggacggcagctgagccttatgaagctaggggtccaggttccctgcctcttgggcctggtc
caccaaccaactatggccctggccactgtgcccagcaggtctcctatcctgatcccaccccagaaggtaaccacgtg
ITRL-spCas9-gRNA12(NFKB):
(SEQ ID NO: 31)
ctagttattaatagtaatcaattacggggtcattagttcatagcccatatatggagttccgcgttacataacttacggtaaat
ggcccgcctggctgaccgcccaacgacccccgcccattgacgtcaataatgacgtatgttcccatagtaacgtcaatagggacttt
ccattgacgtcaatgggtggagtatttacggtaaactgcccacttggcagtacatcaagtgtatcatatgccaagtacgccccctatt
gacgtcaatgacggtaaatggcccgcctggcattatgcccagtacatgaccttatgggactttcctacttggcagtacatctacgtat
tagtcatcgctattaccatggtgatgcggttttggcagtacatcaatgggcgtggatagcggtttgactcacggggatttccaagtct
ccaccccattgacgtcaatgggagtttgttttgcaccaaaatcaacgggactttccaaaatgtcgtaacaactccgccccattgacg
caaatgggcggtaggcgtgtacggtgggaggtctatataagcagagctcgtttagtgaaccgtcagatcgcctggagacgccatc
cacgctgttttgacctccatagaagacaccgggaccgatccagcctccgcggattcgaatcccggccgggaacggtgcattgga
acgcggattccccgtgccaagagtgacgtaagtaccgcctatagagtctataggcccacaaaaaatgctttcttcttttaatatactttt
ttgtttatcttatttctaatactttccctaatctctttctttcagggcaataatgatacaatgtatcatgcctctttgcaccattctaaagaata
acagtgataatttctgggttaaggcaatagcaatatttctgcatataaatatttctgcatataaattgtaactgatgtaagaggtttcatatt
gctaatagcagctacaatccagctaccattctgcttttattttatggttgggataaggctggattattctgagtccaagctaggccctttt
gctaatcatgttcatacctcttatcttcctcccacagctcctgggcaacgtgctggtctgtgtgctggcccatcactttggcaaagaatt
gggattcgaacatcgattgaattcatggactataaggaccacgacggagactacaaggatcatgatattgattacaaagacgatga
cgataagatggccccaaagaagaagcggaaggtcggtatccacggagtcccagcagccgacaagaagtacagcatcggcctg
gacatcggcaccaactctgtgggctgggccgtgatcaccgacgagtacaaggtgcccagcaagaaattcaaggtgctgggcaa
caccgaccggcacagcatcaagaagaacctgatcggagccctgctgttcgacagcggcgaaacagccgaggccacccggctg
aagagaaccgccagaagaagatacaccagacggaagaaccggatctgctatctgcaagagatcttcagcaacgagatggccaa
ggtggacgacagcttcttccacagactggaagagtccttcctggtggaagaggataagaagcacgagcggcaccccatcttcgg
caacatcgtggacgaggtggcctaccacgagaagtaccccaccatctaccacctgagaaagaaactggtggacagcaccgaca
aggccgacctgcggctgatctatctggccctggcccacatgatcaagttccggggccacttcctgatcgagggcgacctgaaccc
cgacaacagcgacgtggacaagctgttcatccagctggtgcagacctacaaccagctgttcgaggaaaaccccatcaacgccag
cggcgtggacgccaaggccatcctgtctgccagactgagcaagagcagacggctggaaaatctgatcgcccagctgcccggc
gagaagaagaatggcctgttcggaaacctgattgccctgagcctgggcctgacccccaacttcaagagcaacttcgacctggcc
gaggatgccaaactgcagctgagcaaggacacctacgacgacgacctggacaacctgctggcccagatcggcgaccagtacg
ccgacctgtttctggccgccaagaacctgtccgacgccatcctgctgagcgacatcctgagagtgaacaccgagatcaccaagg
cccccctgagcgcctctatgatcaagagatacgacgagcaccaccaggacctgaccctgctgaaagctctcgtgcggcagcag
ctgcctgagaagtacaaagagattttcttcgaccagagcaagaacggctacgccggctacattgacggcggagccagccagga
agagttctacaagttcatcaagcccatcctggaaaagatggacggcaccgaggaactgctcgtgaagctgaacagagaggacct
gctgcggaagcagcggaccttcgacaacggcagcatcccccaccagatccacctgggagagctgcacgccattctgcggcgg
caggaagatttttacccattcctgaaggacaaccgggaaaagatcgagaagatcctgaccttccgcatcccctactacgtgggccc
tctggccaggggaaacagcagattcgcctggatgaccagaaagagcgaggaaaccatcaccccctggaacttcgaggaagtg
gtggacaagggcgcttccgcccagagcttcatcgagcggatgaccaacttcgataagaacctgcccaacgagaaggtgctgcc
caagcacagcctgctgtacgagtacttcaccgtgtataacgagctgaccaaagtgaaatacgtgaccgagggaatgagaaagcc
cgccttcctgagcggcgagcagaaaaaggccatcgtggacctgctgttcaagaccaaccggaaagtgaccgtgaagcagctga
aagaggactacttcaagaaaatcgagtgcttcgactccgtggaaatctccggcgtggaagatcggttcaacgcctccctgggcac
ataccacgatctgctgaaaattatcaaggacaaggacttcctggacaatgaggaaaacgaggacattctggaagatatcgtgctga
ccctgacactgtttgaggacagagagatgatcgaggaacggctgaaaacctatgcccacctgttcgacgacaaagtgatgaagc
agctgaagcggcggagatacaccggctggggcaggctgagccggaagctgatcaacggcatccgggacaagcagtccggca
agacaatcctggatttcctgaagtccgacggcttcgccaacagaaacttcatgcagctgatccacgacgacagcctgacctttaaa
gaggacatccagaaagcccaggtgtccggccagggcgatagcctgcacgagcacattgccaatctggccggcagccccgcca
ttaagaagggcatcctgcagacagtgaaggtggtggacgagctcgtgaaagtgatgggccggcacaagcccgagaacatcgtg
atcgaaatggccagagagaaccagaccacccagaagggacagaagaacagccgcgagagaatgaagcggatcgaagaggg
catcaaagagctgggcagccagatcctgaaagaacaccccgtggaaaacacccagctgcagaacgagaagctgtacctgtact
acctgcagaatgggcgggatatgtacgtggaccaggaactggacatcaaccggctgtccgactacgatgtggaccatatcgtgc
ctcagagctttctgaaggacgactccatcgacaacaaggtgctgaccagaagcgacaagaaccggggcaagagcgacaacgt
gccctccgaagaggtcgtgaagaagatgaagaactactggcggcagctgctgaacgccaagctgattacccagagaaagttcg
acaatctgaccaaggccgagagaggcggcctgagcgaactggataaggccggcttcatcaagagacagctggtggaaacccg
gcagatcacaaagcacgtggcacagatcctggactcccggatgaacactaagtacgacgagaatgacaagctgatccgggaag
tgaaagtgatcaccctgaagtccaagctggtgtccgatttccggaaggatttccagttttacaaagtgcgcgagatcaacaactacc
accacgcccacgacgcctacctgaacgccgtcgtgggaaccgccctgatcaaaaagtaccctaagctggaaagcgagttcgtgt
acggcgactacaaggtgtacgacgtgcggaagatgatcgccaagagcgagcaggaaatcggcaaggctaccgccaagtactt
cttctacagcaacatcatgaactttttcaagaccgagattaccctggccaacggcgagatccggaagcggcctctgatcgagacaa
acggcgaaaccggggagatcgtgtgggataagggccgggattttgccaccgtgcggaaagtgctgagcatgccccaagtgaat
atcgtgaaaaagaccgaggtgcagacaggcggcttcagcaaagagtctatcctgcccaagaggaacagcgataagctgatcgc
cagaaagaaggactgggaccctaagaagtacggcggcttcgacagccccaccgtggcctattctgtgctggtggtggccaaagt
ggaaaagggcaagtccaagaaactgaagagtgtgaaagagctgctggggatcaccatcatggaaagaagcagcttcgagaag
aatcccatcgactttctggaagccaagggctacaaagaagtgaaaaaggacctgatcatcaagctgcctaagtactccctgttcga
gctggaaaacggccggaagagaatgctggcctctgccggcgaactgcagaagggaaacgaactggccctgccctccaaatat
gtgaacttcctgtacctggccagccactatgagaagctgaagggctcccccgaggataatgagcagaaacagctgtttgtggaac
agcacaagcactacctggacgagatcatcgagcagatcagcgagttctccaagagagtgatcctggccgacgctaatctggaca
aagtgctgtccgcctacaacaagcaccgggataagcccatcagagagcaggccgagaatatcatccacctgtttaccctgaccaa
tctgggagcccctgccgccttcaagtactttgacaccaccatcgaccggaagaggtacaccagcaccaaagaggtgctggacgc
caccctgatccaccagagcatcaccggcctgtacgagacacggatcgacctgtctcagctgggaggcgacaaaaggccggcg
gccacgaaaaaggccggccaggcaaaaaagaaaaagtgactcgagagatctacgggtggcatccctgtgacccctccccagt
gcctctcctggccctggaagttgccactccagtgcccaccagccttgtcctaataaaattaagttgcatcattttgtctgactaggtgt
ccttctataatattatggggtggaggggggtggtatggagcaaggggcaagttgggaagacaacctgtagggcctgcggggtct
attgggaaccaagctggagtgcagtggcacaatcttggctcactgcaatctccgcctcctgggttcaagcgattctcctgcctcagc
ctcccgagttgttgggattccaggcatgcatgaccaggctcagctaatttttgtttttttggtagagacggggtttcaccatattggcca
ggctggtctccaactcctaatctcaggtgatctacccaccttggcctcccaaattgctgggattacaggcgtgaaccactgctccctt
ccctgtccttctgattttgtaggtaaccagtttggttaattaacgtaccgagggcctatttcccatgattccttcatatttgcatatacgata
caaggctgttagagagataattagaattaatttgactgtaaacacaaagatattagtacaaaatacgtgacgtagaaagtaataatttc
ttgggtagtttgcagttttaaaattatgttttaaaatggactatcatatgcttaccgtaacttgaaagtatttcgatttcttggctttatatatct
tgtggaaaggacgaaacaccgcgagggaccagccaagatcggttttagagctagaaatagcaagttaaaataaggctagtccgtt
atcaacttgaaaaagtggcaccgagtcggtgctttttttacggatccgacgccgccatctctaggcccgcgccggccccctcgcac
agacttgtgggagaagctcggctactcccctgccccggttaatttgcatataatatttcctagtaactatagaggcttaatgtgcgata
aaagacagataatctgttctttttaatactagctacattttacatgataggcttggatttctataagagatacaaatactaaattattatttta
aaaaacagcacaaaaggaaactcaccctaactgtaaagtaattgtgtgttttgagactataaatatcccttggagaaaagccttgtttg
agactgtgggcatgagcacggttttagagctagaaatagcaagttaaaataaggctagtccgttatcaacttgaaaaagtggcacc
gagtcggtgcttttttgaattacgttacataacttacggtaaccacgtg

The second aspect of the present disclosure provides a recombinant adeno-associated virus vector system, including a first recombinant adeno-associated virus vector and a second recombinant adeno-associated virus vector. The first recombinant adeno-associated virus vector includes the first packaging and expression vector as described above, and the second recombinant adeno-associated virus vector includes the second packaging and expression vector as described above.

The third aspect of the present disclosure provides a host cell, wherein the host cell is transduced with the recombinant adeno-associated virus vector system as described above.

In the present disclosure, the host cell is human-derived or from other mammals.

The fourth aspect of the present disclosure provides a use of the construction system as described above in the preparation of gene therapy drugs and/or vaccines, or for gene editing.

The fifth aspect of the present disclosure provides a gene therapy drug, wherein the gene therapy drug includes the construction system as described above.

The present disclosure further provides a method for constructing an expression vector of a large gene, including constructing the positive strand expression vector and negative strand expression vector of the construction system as described above.

The sixth aspect of the present disclosure provides a gene editing method, wherein the method includes using the construction system as described above or the recombinant adeno-associated virus vector system as described above. Specifically, the method includes constructing the construction system as described above, wherein the complete target gene expression cassette corresponding to the construction system includes CRISPR/CAS9 and gRNA, and introducing the construction system into a host containing a gene to be edited.

The seventh aspect of the present disclosure provides a genetic recombination method, wherein the method includes using the construction system as described above or the recombinant adeno-associated virus vector system as described above. Specifically, the method includes constructing the construction system as described above, wherein the complete target gene expression cassette corresponding to the construction system includes EGFP and luciferase, and introducing the construction system into a host.

The implementation of the present disclosure is described below through specific examples. Those skilled in the art may readily appreciate other advantages and efficacies of the present disclosure from the content disclosed herein.

It should be understood that the protection scope of the present disclosure is not limited to the specific embodiments described below. It should also be understood that the terminology used in the examples is for describing specific embodiments and not intended to limit the scope of protection. For experimental methods without specified conditions in the following examples, they are generally performed under conventional conditions or according to conditions recommended by manufacturers.

It should be understood that when a numerical range is provided in the examples, unless otherwise specified, any value between and including the two endpoints of the range may be adopted. Unless otherwise defined, all technical and scientific terms used herein have the same meanings as commonly understood by those skilled in the art. In addition to the specific methods, equipment, and materials used in the examples, any methods, equipment, and materials similar or equivalent to those in the prior art may also be used to implement the present disclosure based on the knowledge of the prior art possessed by those skilled in the art and the disclosures herein.

### EXAMPLE 1

As shown in FIG. 1, the ITR genetic element (DtrsA′B′BC′CA) includes two palindromic arms (B-B′ and C-C′), one long palindromic stem (A-A′), and a D region.

ITR 145 bp Sequence:
(SEQ ID NO: 32)
aggaacccctagtgatggagttggccactccctctctgcgcgctcgctc
gctcactgaggccgggcgaccaaaggtcgcccgacgcccgggctttgcc
cgggcggcctcagtgagcgagcgagcgcgcagagagggagtggccaa

In the present disclosure, the ITR is modified, and a modified ITR sequence (DttRR) is obtained. The modified ITR sequence (DttRR) includes only the Rep binding element (RBE) and Rep terminal resolution site (trs).

DttRR Sequence:
(SEQ ID NO: 33)
aggaacccctagtgatggagttggagttggctgcgcgctcgctcgctcac
tgctgcgcgctcgctcgctcactg
D Sequence:
(SEQ ID NO: 35)
aggaacccctagtgatggag
Trs Sequence:
agttgg
RBE Sequence:
(SEQ ID NO: 36)
ctgcgcgctcgctcgctcactg

In the present disclosure, the ITR is modified, and a modified ITR sequence (DtrsA′B′) is obtained. The modified ITR sequence (DtrsA′B′) is composed of a D element (i.e., D sequence), a trs sequence, an A′ sequence, and a B′ sequence of an AAV ITR element.

DtrsA'B' Sequence:
(SEQ ID NO: 34)
aggaacccctagtgatggagttggccactccctctctgcgcgctcgctcg
ctcactgaggccgggcgaccaaa

### Example 2: Construction of pFBD-DttRR-3.1kb stuff1-CEP-2.8kb stuff2-R′R′t′t′D′ Recombinant Plasmid

This example describes the construction of the pFBD-DttRR-3.1kb stuff1-CEP-2.8kb stuff2-R′R′t′t′D′ recombinant plasmid, where CEP is the abbreviation for CMV-EGFP-PolyA. The construction method includes the following steps:

2.1 Construction of pFBD-DttRR-3.1kb stuff1-CEP-ITR Recombinant Plasmid

Primer 1 and Primer 2 were denatured and subsequently annealed to yield double-stranded oligodeoxynucleotide (OligodsDNA) with sticky ends.

Primer 1:
(SEQ ID NO: 1)
5'-CAGGAACCCCTAGTGATGGAGTTGGAGTTGGCTGCGCGCTCGCTCG
CTCACTGCTGCGCGCTCGCTCGCTCACTGA-3'
Primer 2:
(SEQ ID NO: 2)
5'-CGCGTCAGTGAGCGAGCGAGCGCGCAGCAGTGAGCGAGCGAGCGCG
CAGCCAACTCCAACTCCATCACTAGGGGTTCCTGGTAC-3'

The denaturation and annealing reaction system for Primer 1 and Primer 2 is listed in Table 1.

TABLE 1

| Reagent | Volume |
|---|---|
| 10 × PCR Buffer | 2 μL |
| F(10 uM) Primer 1 | 2 μL |
| R(10 uM) Primer 2 | 2 μL |
| ddH₂O | 14 μL |

The program for the denaturation and annealing reaction was as follows: 95 °C for 30 min, followed by annealing by cooling to room temperature.

The PCR program was set as follows: 95 °C for 4 min; then 25 cycles of 95 °C for 10 s, 56 °C for 30 s, and 72 °C for 3.5 min.

The obtained OligodsDNA with sticky ends was ligated with a pFastBac dual-ITR-EGFP plasmid that has been digested with KpnI and MluI, to yield a pFBD-DttRR-CEP-ITR recombinant plasmid.

The construction method and map of the pFastBac dual-ITR-EGFP plasmid are described in Literature 1 (Li T M et al., Preparation of a novel AAV-ITR gene expression mini vector in Sf9 insect cells via baculovirus, Chin J Biotech, 2015, 31(8), Section 1.2.1 on page 1232).

2.2 Construction of pFBD-DttRR-CEP-R′R′t′t′D′ Recombinant Plasmid

Primer 3 and Primer 4 were denatured and subsequently annealed to yield double-stranded oligodeoxynucleotide (OligodsDNA) with sticky ends.

Primer 3:
(SEQ ID NO: 3)
5'-GACCGCAGTGAGCGAGCGAGCGCGCAGCAGTGAGCGAGCGAGCGC
GCAGCCAACTCCAACTCCATCACTAGGGGTTCCTA-3'
Primer 4:
(SEQ ID NO: 4)
5'-AGCTTAGGAACCCCTAGTGATGGAGTTGGAGTTGGCTGCGCGCTCG
CTCGCTCACTGCTGCGCGCTCGCTCGCTCACTGCG-3'

The denaturation and annealing reaction system for Primer 3 and Primer 4 is listed in Table 2.

TABLE 2

| Reagent | Volume |
|---|---|
| 10 × PCR Buffer | 2 μL |
| F(10 uM) Primer 3 | 2 μL |
| R(10 uM) Primer 4 | 2 μL |
| ddH₂O | 14 μL |

The program for the denaturation and annealing reaction was set as follows: 95 °C for 30 min, followed by annealing by cooling to room temperature.

The obtained double-stranded oligodeoxynucleotide (OligodsDNA) with sticky ends was ligated with the pFBD-DttRR-CEP-ITR recombinant plasmid from Step 2.1, which had been digested with RsrII and HindIII, to yield a pFBD-DttRR-CEP-R′R′t′t′D′ recombinant plasmid.

2.3 Construction of pFBD-DttRR-3.1kb stuff1-CEP-R′R′t′t′D′ Recombinant Plasmid

Using SaCas9-gene as a template, PCR amplification was performed with Primer 5 and Primer 6 to yield a 3.1kb stuff1 fragment with sticky ends.

Primer 5:
(SEQ ID NO: 5)
5'-TCACTGACGCGTCCTGAACGACCTGAACAATCT-3'
Primer 6:
(SEQ ID NO: 6)
5'-AACTAGACGCGTATCCTTAGCGAGGGGGCAGGG-3'

The PCR reaction system is listed in Table 3.

TABLE 3

| Reagent | Volume | |
|---|---|---|
| KOD-Plus-Neo | 2.5 | μL |
| 10 × PCR Buffer | 5 | μL |
| 25 mM MgSO4 | 5 | μL |
| 2 mM dNTPs | 5 | μL |
| F(10 uM) Primer 5 | 0.6 | μL |
| R(10 uM) Primer 6 | 0.6 | μL |
| SaCas9-gene template | 1-50 | ng |

| | |
|---|---|
| ddH₂O | Add water to 50 μL |

The PCR program was set as follows: 95 °C for 4 min; then 25 cycles of 95 °C for 10 s, 56 °C for 30 s, and 72 °C for 3 min.

The obtained 3.1kb stuff1 fragment with sticky ends was ligated with the pFBD-DttRR-CEP-R′R′t′t′D′ recombinant plasmid from Step 2.2, which had been digested with MluI and treated with alkaline phosphatase, to yield a pFBD-DttRR-3.1kb stuff1-CEP-R′R′t′t′D′ recombinant plasmid. [00142]2.4 Construction of pFBD-DttRR-3.1kb stuff1-CEP-2.8kb stuff2-R′R′t′t′D′ Recombinant Plasmid

Using Gli-1 as a template, PCR amplification was performed with Primer 7 and Primer 8, to yield a 2.8kb stuff2 fragment with sticky ends.

Primer 7:
(SEQ ID NO: 7)
5'-TTTGTAGGTAACCAACCGTAATCCGGACCTCACC-3'
Primer 8:
(SEQ ID NO: 8)
5'-GCACGTGGTTACCAGGGTACAAGGCAGGGCCTGC-3'

The PCR reaction system is listed in Table 4.

TABLE 4

| Reagent | Volume | |
|---|---|---|
| KOD-Plus-Neo | 2.5 | μL |
| 10 × PCR Buffer | 5 | μL |
| 25 mM MgSO4 | 5 | μL |
| 2 mM dNTPs | 5 | μL |
| F(10 uM) Primer 7 | 0.6 | μL |
| R(10 uM) Primer 8 | 0.6 | μL |
| Gli-1-gene template | 1-50 | ng |

| | |
|---|---|
| ddH₂O | Add water to 50 μL |

The PCR program was set as follows: 95 °C for 4 min; then 25 cycles of 95 °C for 10 s, 56 °C for 30 s, and 72 °C for 3 min.

The obtained 2.8kb stuff2 fragment with sticky ends was ligated with the pFBD-DttRR-3.1kb stuff1-CEP-R′R′t′t′D′ recombinant plasmid from Step 2.3, which had been digested with BstEII and treated with alkaline phosphatase, to yield a pFBD-DttRR-3.1kb stuff1-CEP-2.8kb stuff2-R′R′t′t′D′ recombinant plasmid. The nucleotide sequence of DttRR-3.1kb stuff1-CEP-2.8kb stuff2-R′R′t′t′D′ is shown as SEQ ID NO:29.

### Example 3: Construction of pFBD-DttRR-2.4kb stuff1-CLP-1.8kb stuff2-R′R′t′t′D′ Recombinant Plasmid

The construction method of the pFBD-DttRR-2.4kb stuff1-CLP-1.8kb stuff2-R′R′t′t′D′ (where CLP is the abbreviation for CMV-Luciferase-PolyA) recombinant plasmid includes the following steps:

3.1 Construction of pFBD-DttRR-CLP-R′R′t′t′D′ Recombinant Plasmid

Using the luciferase gene as a template, PCR amplification was performed with Primer 9 and Primer 10 to yield a luciferase fragment with sticky ends.

Primer 9:
(SEQ ID NO: 9)
5'-TCGAACATCGATATGGAAGATGCCAAAAACATT-3'
Primer 10:
(SEQ ID NO: 10)
5'-AGATCTCTCGAGTTACACGGCGATCTTGCCGCC-3'

The PCR reaction system is listed in Table 5.

TABLE 5

| Reagent | Volume | |
|---|---|---|
| KOD-Plus-Neo | 2.5 | μL |
| 10 × PCR Buffer | 5 | μL |
| 25 mM MgSO4 | 5 | μL |
| 2 mM dNTPs | 5 | μL |
| F (10 uM) Primer 9 | 0.6 | μL |
| R (10 uM) Primer 10 | 0.6 | μL |
| Luciferase gene template | 1-50 | ng |

| | |
|---|---|
| ddH₂O | Add water to 50 μL |

The PCR program was set as follows: 95 °C for 4 min; then 25 cycles of 95 °C for 10 s, 56 °C for 30 s, and 72 °C for 3 min.

The obtained luciferase fragment with sticky ends was ligated with the pFBD-DttRR-CEP-R′R′t′t′D′ recombinant plasmid from Step 2.2 of Example 2, which had been digested with ClaI and XhoI, to yield a pFBD-DttRR-CLP-R′R′t′t′D′ recombinant plasmid.

3.2 Construction of pFBD-DttRR-2.4kb stuff1-CLP-R′R′t′t′D′ Recombinant Plasmid

Using the SaCas9 gene as a template, PCR amplification was performed with Primer 11 and Primer 12, to yield a 2.4kb stuff1 with sticky ends.

Primer 11:
(SEQ ID NO: 11)
5'-TCACTGACGCGTGGCTGTTCAAAGAGGCCAACGT-3'
Primer 12:
(SEQ ID NO: 12)
5'-AACTAGACGCGTTCCAACTTTGTCCCAGCGCCGA-3'

The PCR reaction system is listed in Table 6.

TABLE 6

| Reagent | Volume | |
|---|---|---|
| KOD-Plus-Neo | 2.5 | μL |
| 10 × PCR Buffer | 5 | μL |
| 25 mM MgSO₄ | 5 | μL |
| 2 mM dNTPs | 5 | μL |
| F(10 uM) Primer 11 | 0.6 | μL |
| R(10 uM) Primer 12 | 0.6 | μL |
| SaCas9 gene template | 1-50 | ng |

| | |
|---|---|
| ddH₂O | Add water to 50 μL |

The PCR program was set as follows: 95 °C for 4 min; then 25 cycles of 95 °C for 10 s, 56 °C for 30 s, and 72 °C for 3 min.

The obtained 2.4kb stuff1 with sticky ends was ligated with the pFBD-DttRR-CLP-R′R′t′t′D′ recombinant plasmid from Step 3.1 of this example, which had been digested with MluI and treated with alkaline phosphatase, to yield a pFBD-DttRR-2.4kb stuff1-CLP-R′R′t′t′D′ recombinant plasmid.

3.3 Construction of pFBD-DttRR-2.4kb stuff1-CLP-1.8kb stuff2-R′R′t′t′D′ Recombinant Plasmid

Using the Gli-1 gene as a template, PCR amplification was performed with Primer 13 and Primer 14 to yield a 1.8kb stuff2 fragment with sticky ends.

Primer 13:
(SEQ ID NO: 13)
5'-TTTGTAGGTAACCTGCCAGCTGAAGTCAGAGCTG-3'
Primer 14:
(SEQ ID NO: 14)
5'-GCACGTGGTTACCTTCTGGGGTGGGATCAGGATA-3'

The PCR reaction system is listed in Table 7.

TABLE 7

| Reagent | Volume | |
|---|---|---|
| KOD-Plus-Neo | 2.5 | μL |
| 10 × PCR Buffer | 5 | μL |
| 25 mM MgSO₄ | 5 | μL |
| 2 mM dNTPs | 5 | μL |
| F(10 uM) Primer 13 | 0.6 | μL |
| R(10 uM) Primer 14 | 0.6 | μL |
| Gli-1 gene template | 0.6 | μL |

| | |
|---|---|
| ddH₂O | Add water to 50 μL |

The PCR program was set as follows: 95 °C for 4 min; then 25 cycles of 95 °C for 10 s, 56 °C for 30 s, and 72 °C for 2 min.

The obtained 1.8kb stuff2 fragment with sticky ends was ligated with the pFBD-DttRR-2.4kb stuff1-CLP-R′R′t′t′D′ recombinant plasmid from Step 3.2 of this example, which had been digested with BstEII and treated with alkaline phosphatase, to yield the pFBD-DttRR-2.4kb stuff1-CLP-1.8kb stuff2-R′R′t′t′D′ recombinant plasmid. The nucleotide sequence of DttRR-2.4kb stuff1-CLP-1.8kb stuff2-R′R′t′t′D′ is shown as SEQ ID NO:30.

### Example 4: pFBD-Expression Cassette 1 5.0kb-ITRL-CMV-spCas9-PolyA-U6-gRNA1/2(NFkB)-RXH Recombinant Plasmid

This example describes the construction method for pFBD-expression cassette 1 5.0kb-ITRL-CMV-spCas9-PolyA-U6-gRNA1/2(NF-KB)-RXH, including the following steps:

4.1 Construction of pFBL-CEP-RXH Recombinant Plasmid

Primer 15 and Primer 16 were denatured and subsequently annealed to yield double-stranded oligodeoxynucleotide (OligodsDNA) with sticky ends.

Primer 15:
(SEQ ID NO: 15)
5'-GACCGTAATCTCTAGATTGAGGAACAA-3'
Primer 16:
(SEQ ID NO: 16)
5'-AGCTTTGTTCCTCAATCTAGAGATTACG-3'

The denaturation and annealing reaction system for Primer 15 and Primer 16 is listed in Table 8.

TABLE 8

| Reagent | Volume |
|---|---|
| 10 × PCR Buffer | 2 μL |
| F(10 uM) Primer 15 | 2 μL |
| R(10 uM) Primer 16 | 2 μL |
| ddH₂O | 14 μL |

The program for the denaturation and annealing reaction was set as follows: 95 °C for 30 min, followed by annealing by cooling to room temperature.

The obtained OligodsDNA with sticky ends was ligated with the pFastBacdual-ITR-EGFP plasmid that has been digested with RsrII and HindIII to yield a pFBD-ITRL-CEP-RXH recombinant plasmid.

4.2 Construction of pFBD-ITRL-CMV-spCas9-polyA-RXH Recombinant Plasmid

Using spCas9 as a template, PCR amplification was performed with Primer 17 and Primer 18 to yield an spCas9 fragment with sticky ends.

Primer 17:
(SEQ ID NO: 17)
5'-TCGAACGAATTCATGGACTATAAGGACCACGAC-3'
Primer 18:
(SEQ ID NO: 18)
5'-AGATCTCTCGAGTCACTTTTTCTTTTTTGCCTG-3'

The PCR reaction system is listed in Table 9.

TABLE 9

| Reagent | Volume | |
|---|---|---|
| KOD-Plus-Neo | 2.5 | μL |
| 10 × PCR Buffer | 5 | μL |
| 25 mM MgSO4 | 5 | μL |
| 2 mM dNTPs | 5 | μL |
| F(10 uM) Primer 17 | 0.6 | μL |
| R(10 uM) Primer 18 | 0.6 | μL |
| spCas9 gene template | 1-50 | ng |

| | |
|---|---|
| ddH₂O | Add water to 50 μL |

The PCR program is set as follows: 95 °C for 4 min; then 25 cycles of 95 °C for 10 s, 56 °C for 30 s, and 72 °C for 4 min.

The obtained spCas9 fragment with sticky ends was ligated with the pFBD-ITRL-CEP-RXH recombinant plasmid from Step 4.1 of this example, which had been digested with EcoRI and XhoI, to yield a pFBD-ITRL-CMV-spCas9-polyA-RXH recombinant plasmid.

4.3 Construction of pFBD-ITRL-CMV-spCas9-polyA-U6-gRNA1/2(NFkB)-RXH Recombinant Plasmid

Using U6-gRNA1/2(NF-KB) as a template, PCR amplification was performed with Primer 19 and Primer 20 to yield a U6-gRNA1/2(NF-KB) expression cassette with sticky ends.

Primer 19:
(SEQ ID NO: 19)
5'-TTTGTAGGTAACCAGTTTGGTTAATTAACGTACCGA-3'
Primer 20:
(SEQ ID NO: 20)
5'-GCACGTGGTTACCGTAAGTTATGTAACGTAATTCAA-3'

The PCR reaction system is listed in Table 10.

TABLE 10

| Reagent | Volume | |
|---|---|---|
| KOD-Plus-Neo | 2.5 | μL |
| 10 × PCR Buffer | 5 | μL |
| 25 mM MgSO₄ | 5 | μL |
| 2 mM dNTPs | 5 | μL |
| F(10 uM) Primer 19 | 0.6 | μL |
| R(10 uM) Primer 20 | 0.6 | μL |
| U6-gRNA1/2(NF-KB) gene template | 1-50 | ng |

| | |
|---|---|
| ddH₂O | Add water to 50 μL |

The PCR program was set as follows: 95 °C for 4 min; then 25 cycles of 95 °C for 10 s, 56 °C for 30 s, and 72 °C for 1 min.

The obtained U6-gRNA1/2(NF-KB) expression cassette with sticky ends was ligated with the pFBD-ITRL-CMV-spCas9-polyA-RXH recombinant plasmid from Step 4.2 of this example, which had been digested with BstEII and treated with alkaline phosphatase, to yield a pFBD-ITRL-CMV-spCas9-polyA-U6-gRNA1/2(NF-KB)-RXH recombinant plasmid.

4.4 Construction of pFBD-expression cassette 1 5.0kb-ITRL-CMV-spCas9-PolyA-U6-gRNA1/2(NFkB)-RXH Recombinant Plasmid

Using CMV-spCas9-PolyA-U6-gRNA1/2(NF-KB) as a template, PCR amplification was performed with Primer 21 and Primer 22 to yield a 15.0kb expression cassette with sticky ends.

Primer 21:
(SEQ ID NO: 21)
5'-TCTCCCGGTACCATGATCAAGTTCCGGGGCCAC-3'
Primer 22:
(SEQ ID NO: 22)
5'-CATGGTGCTAGCGTAAGTTATGTAACGTAATTC-3'

The PCR reaction system is listed in Table 11.

TABLE 11

| Reagent | Volume | |
|---|---|---|
| KOD-Plus-Neo | 2.5 | μL |
| 10 × PCR Buffer | 5 | μL |
| 25 mM MgSO₄ | 5 | μL |
| 2 mM dNTPs | 5 | μL |
| F(10 uM)Primer21 | 0.6 | μL |
| R(10 uM)Primer22 | 0.6 | μL |
| CMV-spCas9-PolyA-U6-gRNA1/2(NF-KB) | 1-50 | ng |
| template | | |

| | |
|---|---|
| ddH₂O | Add water to 50 μL |

The PCR program was set as follows: 95 °C for 4 min; then 25 cycles of 95 °C for 10 s, 56 °C for 30 s, and 72 °C for 5 min.

The obtained 15.0kb expression cassette with sticky ends was ligated with the pFBD-ITRL-CMV-spCas9-polyA-U6-gRNA1/2(NF-KB)-RXH recombinant plasmid from Step 4.3 of this example, which had been digested with KpnI and NheI, to yield a pFBD-expression cassette 1 5.0kb-ITRL-CMV-spCas9-PolyA-U6-gRNA1/2(NF-KB)-RXH plasmid. The nucleotide sequence of ITRL-spCas9-gRNA12(NFkB) is shown in SEQ ID NO:31.

### Example 5: Virus Packaging

The packaging system for recombinant adeno-associated virus vector for large gene packaging and expression of the present disclosure may be a baculovirus-insect cell packaging system or a mammalian cell packaging system, wherein the insect cells used are the Sf9 cells.

First, one recombinant plasmid expressing both AAV replication protein (Rep) and AAV capsid protein (Cap) or two recombinant plasmids expressing Rep and Cap, respectively, and one plasmid containing the lgAAV DNA genome were individually transformed into E. coli DH10Bac competent cells. Through two rounds of blue-white screening, colonies containing recombinant bacmids were white, while non-recombinant colonies were blue. White colonies were selected for expansion, and recombinant bacmids were then extracted.

Then, using an insect cell transfection reagent, the above two or three recombinant bacmids were transfected into Sf9 insect cells. After 4-5 days, the cell supernatant was collected to obtain the P1 generation recombinant baculoviruses. The P1 recombinant baculoviruses were propagated by infecting Sf9 insect cells twice to obtain the P3 generation recombinant baculoviruses. Viral titer (pfu/mL) of P3 baculoviruses was determined by plaque assay, and calculated as follows: Viral titer (pfu/mL)=(1/dilution factor)×number of plaques×(1/inoculation volume per well).

Finally, two or three types of P3 recombinant baculoviruses were co-infected into Sf9 insect cells to package and produce the lgAAV vector particles. Specific procedures may be performed according to Literature 2 (Urabe M, Ding C, Kotin R M. Insect cells as a factory to produce adeno-associated virus type 2 vectors. Hum Gene Ther. Nov. 1, 2002; 13(16):1935-43. doi:10.1089/10430340260355347. PMID:12427305). The procedures include the following steps:

### 5.1 Preparation of Recombinant Bacmid

1) 100 μL DH10Bac competent cells were thawed slowly on ice for 1-3 min.

2) 500 ng recombinant plasmid DNA from Examples 2, 3, and 4 were added, respectively, followed by gentle mixing.

3) The mixture was incubated on ice for 30 min, heat-shocked at 42 °C for 90 s, and immediately transferred to ice for 3 min.

4) 890 μL LB medium was added, and the mixture was shaken at 225 rpm at 37 °C for 2-3 h.

5) 40 μL of 2% (20 mg/mL) X-gal and 20 μL of 20% (200 mg/mL) IPTG were dropped to the center of a pre-prepared 90 mm KTG-resistant LB agarose plate, which contained kanamycin (Kan) with a final concentration of 50 g/mL, gentamicin (Gen) with a final concentration of 77 g/mL, and tetracycline (Tet) with a final concentration of 10 g/mL. The solution was spread evenly using a sterile spreader and incubated in an oven until the liquid was fully absorbed.

6) 100 μL and 300 μL gradient bacterial cultures were spread onto the KTG-resistant agarose plate.

7) The plate was incubated at 37 °C for 48 h. Two white colonies were picked and streaked onto a fresh KTG-resistant agarose plate, followed by overnight incubation at 37 °C.

8) Two monoclonies were selected for PCR identification. Bacmid was identified by PCR using primer pairs specific for the target gene (F/R) and for pUC/M13 (F/R).

9) PCR-confirmed Bacmid-positive colonies were inoculated into 10 mL LB (Kan+, Gen+, Tet+) and cultured with shaking for 16-18 h. Recombinant bacmid DNA was extracted using the OMEGA kit according to the manufacturer's instructions. Bacmid concentration was measured, aliquoted, and stored at −20 °C.

5.2 Transfection of Recombinant Bacmid Baculovirus into Sf9 Cells

### 5.2.1 Preparation of Transfection Reagent

200 mL 1×HBS solution was prepared as follows: 0.954 g Hepes and 1.754 g NaCl were dissolved in 150 mL sterilized ddH₂O. The pH was adjusted to 7.4 with 1 M NaOH, and the final volume was brought to 200 mL. The solution was then sterile-filtered in a biosafety cabinet and stored at 4 °C.

20 mL PEI was prepared as follows: 0.043 g PEI was dissolved thoroughly in 1 mL anhydrous ethanol, and the final volume was brought to 20 mL with 1×HBS solution. Three freeze-thaw cycles (freezing at −20 °C, thawing at room temperature) were performed, and the obtained solution was stored at −20 °C.

### 5.2.2 Cell Seeding

Sf9 suspension cells were counted and seeded in a 6-well plate at a density of 2×10⁶ cells/mL with 2 mL per well. Cell viability was confirmed to exceed 95%.

### 5.2.3 Transfection (Per Well)

Solution A: 6 μL PEI and 94 μL 1×HBS were mixed and incubated for 4 min.

Solution B: 3 g bacmid DNA (pre-heated at 65 °C for 30 min) was supplemented with 1×HBS to a final volume of 100 μL, followed by gentle mixing.

100 μL Solution A was added to Solution B, mixed, and incubated at room temperature for 30 min. The mixture was added to wells seeded with Sf9 cells, and then cultured for 96 h.

### 5.2.4 Virus Amplification

### 1) Isolation of P1 Generation

After Sf9 cells were confirmed to be in the late stage of infection (96 h), 2 mL virus-containing medium per well was collected into a sterile 15 mL centrifuge tube. The sample was centrifuged at 500×g for 5 min to remove cell debris.

Supernatant was transferred to sterile EP tubes and stored at 4 °C in the dark. For long-term storage, aliquots were stored at −80 °C.

### 2) Amplification of P2 Generation

For infection at an MOI of 0.1, 1.6 mL P1 virus stock was added into 8 mL Sf9 suspension culture (density: 2×10⁶ cells/mL). Cells were cultured at 27 °C for 72 h, collected into sterile 15 mL centrifuge tubes, and centrifuged at 500×g for 5 min. Supernatant was aliquoted into sterile EP tubes as P2 virus stock and stored at 4 °C in the dark or at −80 °C for long-term storage.

### 3) Amplification of P3 Generation

P3 generation was obtained according to the method described as above: for infection at an MOI of 0.1, 200 μL P2 viral stock was added to 10 mL Sf9 suspension culture (density: 2×10⁶ cells/mL). After 72 h incubation, P3 generation was harvested.

Typically, titers of P1 was 1×10⁶-1×10⁷ pfu/mL, while titers of P2 was 1×10⁷-1×10⁸ pfu/mL.

### 4) Virus Packaging

100 mL suspension culture (density: 5×10⁶ cells/mL) was co-transfected with 5 mL each of P3 generation baculovirus expressing Rep, Cap, and the target at an MOI of 1. Cells were harvested after 72 h to obtain recombinant adeno-associated virus vectors, namely: lgAAV-CMV-Luci-7.6kb, lgAAV-CMV-CEP-8.3kb, and lgAAV-spCAS9-gRNA-NFkB-7.0kb.

### Example 6: Virus Purification, Titer Detection, and Characterization

In Example 6, the recombinant adeno-associated virus vectors lgAAV-CMV-Luci-7.6kb, lgAAV-CMV-CEP-8.3kb, and lgAAV-spCAS9-gRNA-NFkB-7.0kb obtained in Example 5 were purified and characterized, including the following steps:

### 6.1 Purification of Viral Suspension

Iodixanol density gradient ultracentrifugation was used to purify the viral suspension from Example 5. Details were as follows:

### 6.1.1 Cell Lysate Preparation

1) After 72 h co-infection, cells were collected by centrifugation at 1000×g for 5 min.

2) Supernatant was discarded. Cells from Step 1) were resuspended in 10 mL Lysis Buffer to obtain a cell suspension. 1 μL of Lysis Buffer was prepared as follows: 8.766 g of NaCl and 6.055 g of Tris were dissolved in 950 mL of DDH₂O. The pH was adjusted to 8.5 with 5 M HCl, and the final volume was brought to 1 μL. Sterile filtration was performed in a biosafety cabinet, and the buffer was stored at 4 °C.

3) The cell suspension from Step 2) was frozen in liquid nitrogen and thawed at 37 °C, repeated 3-4 times until the solution became clear.

4) Supernatant was collected by centrifugation at 4 °C and 5000×g for 30 min. Pellet was resuspended in 4-5 mL PBS and centrifuged again to collect supernatant. These supernatants from centrifugations were combined.

5) DNase (10 μl/mL) and RNase (1 μl/mL) were added to the combined supernatant from Step 4), followed by digestion in a 37 °C water bath for 1 h to obtain cell lysate.

### 6.1.2 Viral Purification by Iodixanol Density Gradient Ultracentrifugation

1) 60% iodixanol was diluted with PBS-MK (1×PBS, 1 mM MgCl₂, 2.5 mM KCl) to 15%, 25%, 40%, and 58% iodixanol gradient phases. Solid NaCl was added to the 15% phase to reach a final concentration of 1 M.

2) 0.5% phenol red solution was added to the 15% and 25% phases at a final concentration of 1.5 μl/mL, and to the 58% phase at a final concentration of 0.5 μl/mL.

3) Using a 1.27×120 mm flat-tip spinal needle, 8 mL 15%, 6 mL 25%, 8 mL 40%, and 5 mL 58% iodixanol dilutions were sequentially added from the bottom of a 39 mL quick-seal ultracentrifuge tube, avoiding bubbles.

4) Cell lysate from Step 6.1.1 was carefully layered onto the iodixanol density gradient solution. The tube was filled (flush with the tube opening). If necessary, Lysis Buffer was added to bring the total volume to capacity. Centrifugation was performed at 69,000 rpm, 18 °C for 1.5 h (BECKMAN COULTER centrifuge, 70Ti rotor).

5) The quick-seal tube was pierced from the bottom. The first 4 mL (58% phase) was discarded, and 6 mL of solution containing the purified virus was collected.

6) Ultrafiltration was performed using Amicon Ultra-4 Centrifugal Filters (Ultracel-100k) at 3000×g for 5 min. Washing with PBS (1×PBS, Solarbio, supplemented with 5M NaCl to a final concentration of 350 mM) was repeated until iodixanol residue concentration was <0.1%. Virus from every 100 mL initial volume was concentrated to approximately 200 L.

7) Glycerol was added to a final concentration of 5%. Sterile filtration was performed, and aliquots were stored at −80 °C to obtain purified virus.

### 6.2 Viral Titer Detection

Real-time Quantitative PCR (RT-PCR) was used to detect the titer of purified recombinant adeno-associated virus vectors. The RT-PCR method included:

1) Plasmid ITR-CMV-EGFP-PolyA-ITR was diluted 10-fold, then serially diluted across 5 gradients to generate a standard curve.

2) Purified recombinant adeno-associated virus vectors were diluted 10-fold, then serially diluted across 4 gradients.

3) Buffer preparation: the buffer solution is prepared in the dark and according to the following formulation:

2× SuperpreMix Plus (SYBR Green) 10 μL

Primer 23:
(SEQ ID NO: 23)
5'-TCCGCGTTACATAACTTACGG-3'
0.3 μL
Primer 24:
(SEQ ID NO: 24)
5'-GGGCGTACTTGGCATATGAT-3'
0.3 μL

ddH₂O 4.4 μL

4) Each reaction was set up in a 20 μL system, consisting of 5 μL of template DNA and 15 μL of the buffer mixture prepared in Step 3. Each dilution gradient was tested in duplicate wells.

5) RT-PCR program (Roche LightCycler):
Stage 1: Pre-denaturation Reps: 1 95 °C 600 s;
Stage 2: Amplification Reps: 40 95 °C 10 s; 55 °C 30 s; 72 °C 30 s;
Stage 3: Melting Curve Reps: 1 95 °C 15 s; 60 °C 60 s; 95 °C 15 s.

### 6.3 Characterization

6.3.1 SDS-PAGE Analysis of Capsid Proteins of Purified Recombinant Adeno-Associated Virus Vectors from 6.2

### 1) Gel Preparation:

i) Resolving Gel (10%): The following components (with a total volume of 9.093 mL) were added to a beaker: 3.69 mL double-distilled water (ddH₂O), 2.97 mL 30% acrylamide/bisacrylamide solution (acrylamide:bis-acrylamide=29:1), 2.25 mL 2M Tris (pH 8.8), 90 μL 10% ammonium persulfate, 90 μL 10% SDS, and 3 μL TEMED. The mixture was stirred and then immediately poured between two glass plates until the liquid level reached 1 cm below the lower edge of the comb. 75% ethanol was slowly layered on topc and the gel was left to polymerize at room temperature. After the polymerization, the 75% ethanol layer was discarded.

ii) Stacking Gel (5%): The following components (with a total volume of 2.357 mL) were added to another beaker: 1.83 mL ddH₂O, 0.39 mL 30% acrylamide/bisacrylamide solution (acrylamide:bis-acrylamide=29:1), 0.75 mL 0.5M Tris (pH 6.8), 30 μL 10% ammonium persulfate, 30 μL 10% SDS, and 2 μL TEMED. The mixture was stirred and then immediately layered over the polymerized resolving gel between the glass plates. The comb was gently inserted. After polymerization, the gel plate was ready for use.

### 2) Sample Preparation:

Viral samples were prepared using the same procedures described for the construction, packaging, and purification of recombinant adeno-associated virus vectors lgAAV-CMV-Luci-7.6kb, lgAAV-CMV-EGFP-8.3kb, and lgAAV-spCAS9-gRNA-NF kB-7.0kb.

Viral sample and 5× loading buffer were mixed at a 2:1 ratio, boiled for 10 min, and centrifuged briefly. The entire sample was loaded for electrophoresis.

### 3) Electrophoresis:

i) The gel plate was vertically mounted on the electrode frame in the electrophoresis tank, with the notched surface facing the electrode frame.

ii) The gel plate and electrode frame were secured in the tank. Electrophoresis buffer was added, and the comb was gently removed.

iii) 15 μL of processed sample was slowly loaded into the wells using a microsyringe.

iv) Electrophoresis was performed at the following voltage conditions: stacking gel: 90 V, resolving gel: 120 V. The run was stopped when the bromophenol blue dye front migrated to approximately 1 cm from the bottom edge of the gel.

v) Staining: Glass plates were separated using a blade or a thin plate. The resolving gel was cut at the stacking/resolving gel interface and transferred to a staining tray. 100 mL staining solution was added, covered, and stained for 1-3 h.

vi) Destaining: The staining solution was discarded. The gel was rinsed with water for several times. The gel was then immersed in fresh water (just covering the gel) and heated in a microwave oven at high power for 2 min. The tray was gently shaken. This was repeated until protein bands were clearly visible.

FIG. 6 shows the SDS-PAGE analysis of capsid proteins for the following recombinant adeno-associated virus vectors: rAAV-EGFP (Lane 1), lgAAV-CMV-Luci-7.6kb (Lane 2), lgAAV-CMV-EGFP-8.3kb (Lane 3), and lgAAV-spCAS9-gRNA-NF kB-7.0RNA-NF kB-7.0kb (Lane 4), wherein MW denotes the protein molecular weight standard.

As shown in FIG. 6, the capsid proteins of the recombinant adeno-associated virus vector lgAAV-CMV-Luci-7.6kb, recombinant adeno-associated virus vector lgAAV-CMV-EGFP-8.3kb, and recombinant adeno-associated virus vector lgAAV-spCAS9-gRNA-NFkB-7.0kb demonstrated no difference in composition compared to capsid proteins of the ordinary recombinant adeno-associated virus vector AAV-EGFP. Capsid proteins of all these recombinant adeno-associated virus vectors included three proteins: VP1, VP2, and VP3, with the stoichiometric ratio of these three proteins being approximately 1:1:10.

### 6.3.2 Characterization of Recombinant Adeno-Associated Virus Vector DNA Genome

### 1) Neutral Agarose Gel Electrophoresis Analysis

i) An agarose solution was prepared by adding a precise amount of agarose powder and a quantified volume of 1×TAE electrophoresis buffer to an Erlenmeyer flask or glass bottle.

ii) The flask neck was loosely covered with Kimwipes paper. For glass bottles, the cap was loosened. The suspension was heated in a boiling water bath or microwave oven until the agarose dissolved.

iii) The clear solution was cooled to 40-50 °C, and 4S Red staining solution was added, followed by immediate pouring. After complete solidification, the gel was placed in an electrophoresis tank and fully submerged in freshly prepared 1×TAE electrophoresis buffer.

iv) The rAAV-CMV-EGFP-PolyA vector prepared as described above and recombinant adeno-associated virus vectors from Example 5 were used. 10 μL recombinant adeno-associated virus vector stock solution was mixed with 1.1 μL 10×PCR Buffer. The mixture was subjected to PCR amplification using the following program: 95 °C for 5 min, 72 °C for 5 min, 55 °C for 5 min, 37 °C for 5 min, 80 °C for 5 min, 72 °C for 5 min, 55 °C for 5 min, 37 °C for 15 min.

v) 0.2 volume of 6× gel loading buffer was added to the denatured/annealed sample.

vi) The entire sample in 6× gel loading buffer was loaded into the wells. Electrophoresis was performed at 100 V for 50 min.

vii) Imaging: The gel was placed in a gel documentation system, and DNA bands were photographed.

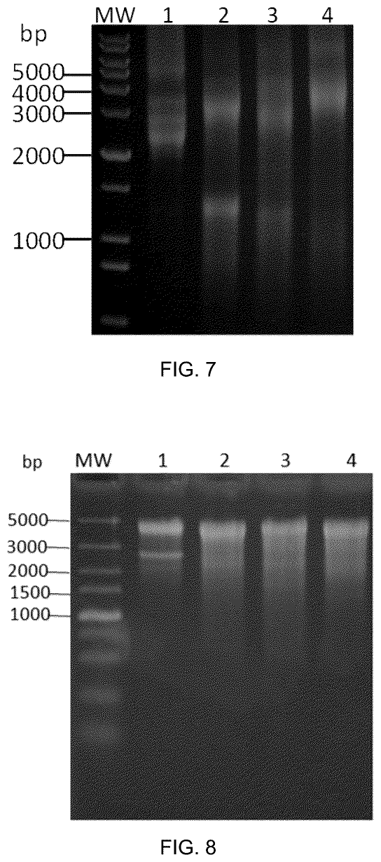

FIG. 7 shows neutral agarose gel electrophoresis results for: rAAV-EGFP vector (Lane 1), lgAAV-CMV-Luci-7.6kb (Lane 2), lgAAV-CMV-EGFP-8.3kb (Lane 3), and lgAAV-spCAS9-gRNA-NF kB-7.0kb (Lane 4), wherein MW denotes the DNA molecular weight standard.

As shown in FIG. 7, Lane 1 corresponded to the rAAV-CMV-EGFP vector. Its DNA primarily consisted of a 4.7 kb double-stranded molecule formed by complementary pairing of positive and negative strands. Because of mismatched regions within the paired strands, the bands appeared diffuse on the gel.

Lane 2 corresponded to the lgAAV-CMV-Luci-7.6kb vector obtained in Example 5, including designed unipolar positive single-stranded single-polarity DNA and negative single-stranded single-polarity DNA molecule (each approximately 4.7 kb in length). Partial complementarity between positive and negative strands resulted in double-stranded DNA with an apparent length shorter than 4.7 kb in neutral agarose gel electrophoresis. Unpaired single-stranded single-polarity DNA molecules appeared as significantly smaller and diffuse bands due to their lower molecular weight.

Lane 3 corresponded to the lgAAV-CMV-EGFP-8.3kb vector obtained in Example 3, including designed positive single-stranded single-polarity DNA and negative single-stranded single-polarity DNA molecule (each approximately 4.7 kb in length). Partial complementarity between positive and negative strands resulted in double-stranded DNA bands with an apparent length shorter than 4.7 kb. Unpaired single-stranded single-polarity DNA appeared as significantly smaller and diffuse bands due to its lower molecular weight.

Lane 4 corresponded to the lgAAV-spCAS9-gRNA-NF kB-6.7kb vector obtained in Example 4, including designed positive single-stranded single-polarity DNA and negative single-stranded single-polarity DNA molecule (each approximately 4.7 kb in length). Partial complementarity between positive and negative strands resulted in double-stranded DNA bands with an apparent length shorter than 4.7 kb. Unpaired single-stranded single-polarity DNA appeared as significantly smaller and diffuse bands due to its lower molecular weight.

### 2) Alkaline Agarose Gel Electrophoresis Analysis

i) Alkaline agarose gel preparation: 0.36 g agarose powder and 27 mL distilled water were added to an Erlenmeyer flask or glass bottle. The flask neck was loosely covered with Kimwipes paper. For glass bottles, the cap was loosened. The suspension was heated in a microwave oven at medium power until the agarose dissolved. The clear solution was equilibrated at 56 °C in a water bath for 5 min. 3 mL 10× alkaline gel electrophoresis buffer (equilibrated at 56 °C for 2 min) was added, mixed, and the mixture was poured immediately to form the gel. After solidification, the gel was placed in an electrophoresis tank and fully submerged in freshly prepared 1× electrophoresis buffer (4 °C).

ii) Sample preparation: The rAAV-CMV-EGFP vector was prepared as described, and three recombinant adeno-associated virus vectors from Example 5 were used. 30 μL viral sample was digested with 3 μL proteinase K (20 mg/mL) at 65 °C for 15 min, centrifuged at 12,000×g for 5 min. 30 μL supernatant was collected and mixed with 6 μL 6× alkaline gel loading buffer.

iii) Electrophoresis: DNA samples were loaded into wells. Electrophoresis was performed at <3.5 V/cm (horizontal electrophoresis tank JY-SPAT: 27 V for 3 h).

iv) Elution: The gel was placed in 400 mL sterile water and gently shaken on a horizontal shaker at 56 rpm for 1 h, with water replacement every 30 min.

v) Staining: The eluted gel was stained with 1×TAE buffer containing 0.5 g/mL ethidium bromide (EB).

vi) Imaging: The gel was placed in a gel documentation system, and DNA bands were photographed.

FIG. 8 shows alkaline agarose gel electrophoresis results for: rAAV-CMV-EGFP (Lane 1), lgAAV-CMV-Luci-7.6kb (Lane 2), lgAAV-CMV-EGFP-8.3kb (Lane 3), and lgAAV-spCAS9-gRNA-NF kB-7.0kb (Lane 4), wherein MW denotes the DNA molecular weight standard.

As shown in FIG. 8, DNA molecules packaged using the rAAV vector were all 4.7 kb single-stranded DNA.

### Example 7: Characterization of Target Gene Expression

In this example, the recombinant adeno-associated virus vectors lgAAV-CMV-Luci-7.6kb, lgAAV-CMV-EGFP-8.3kb, and lgAAV-spCAS9-gRNA-NFkB-7.0kb containing different target genes obtained in Example 5 were expressed and characterized, including the following steps:

7.1 Characterization of Gene Expression in Mouse Eyes Following Injection of lgAAV-CMV-EGFP-8.3kb

1) Intravitreal injection: Mice were anesthetized with 4% chloral hydrate (0.01 mL/g). 0.1 mg/mL proparacaine hydrochloride and 5 mg/mL compound tropicamide eye drops were applied to the eyes for local anesthesia and mydriasis. The mouse head was positioned to maintain the corneal limbus horizontal. A 31G needle was used to puncture approximately 1 mm posterior to the limbus. lgAAV-CMV-EGFP-8.3kb (1 μL, 1E10 vg/eye) was injected via a Hamilton 33G syringe at the puncture site. The needle was inserted perpendicularly, then tilted slightly, and the solution was slowly injected. After injection, the needle was kept in place for 30 seconds before being quickly withdrawn. Sodium hyaluronate (1 mg/mL) and levofloxacin (4.88 mg/mL) eye drops were applied for corneal moisture and anti-inflammation, respectively.

Control groups were set up as follows: Control Group 1 received an injection of the negative vector alone, Control Group 2 received an injection of the positive vector alone, and Control Group 3 received an injection of the rAAV6-CMV-EGFP vector.

2) Fundus imaging of Mouse Eyes:

Mice were anesthetized with 4% chloral hydrate (0.01 mL/g). Anesthetized mice received compound tropicamide (5 mg/mL) for mydriasis and then sodium hyaluronate (1 mg/mL) for corneal moisture. Line-scanning confocal fundus imaging was performed using a line-scanning confocal fundus imaging system on the mice. A 488 nm solid-state laser was used to excite and observe green fluorescence in the mouse eyes on days 10 and 17 post-injection of the lgAAV-CMV-EGFP-8.3kb recombinant adeno-associated virus vector.

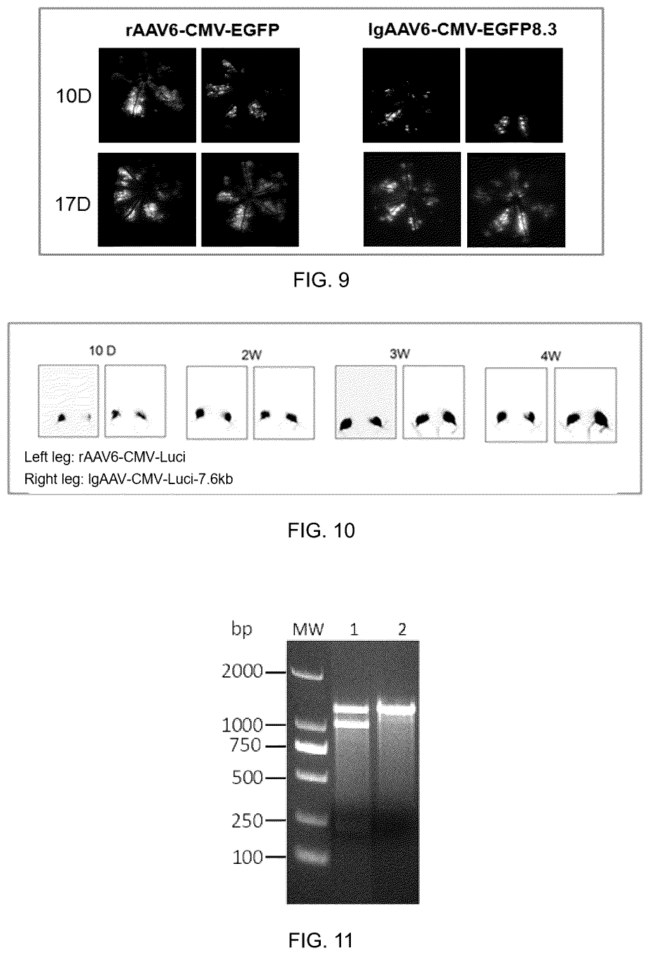

FIG. 9 shows validation results of EGFP expression in mouse eyes following injection with the lgAAV-CMV-EGFP-8.3kb.

As shown in FIG. 9, no fluorescence was observed when positive or negative lgAAV-CMV-EGFP-8.3kb vectors were injected alone, while strong fluorescence was detected with co-injection of positive or negative vectors. This demonstrated that after partial base pairing between the complementary regions of the positive and negative DNA strands, DNA synthesis was initiated from the 3′ end using the paired region as a template. This process resulted in the formation of a complete double-stranded DNA, reconstructing the full-length EGFP expression cassette, and enabling transcription, protein synthesis, and green fluorescence emission.

Thus, the original 4.7 kb single-stranded, single-polarity AAV genomes paired packaged in the positive and negative lgAAV vectors underwent complementary pairing through a 1.1 kb homologous region inside host cells, enabling the synthesis of a full-length 8.3 kb EGFP expression cassette.

7.2 Characterization of Gene Expression in Mouse Muscle Following Injection of lgAAV-CMV-Luci-7.6kb

### 1) Intramuscular Injection:

Mice were anesthetized with 4% chloral hydrate (0.01 mL/g). Anesthetized mice were placed in a supine position on the experimental platform. A syringe was inserted perpendicularly to the semitendinosus muscle (at a 90° angle) to approximately one-quarter of the needle length, and lgAAV-CMV-Luci-7.6kb (100 μL, 5E10 vg/right leg) was injected into the semitendinosus muscle.

Control Group: rAAV6-CMV-Luci was injected into the left leg of the same mice, with the injection dose and method being identical to those used for the lgAAV-CMV-Luci-7.6kb recombinant adeno-associated virus vector.

### 2) Chemiluminescence Imaging:

Mice were anesthetized with 4% chloral hydrate (0.01 mL/g). Anesthetized mice received an intraperitoneal injection of D-luciferin sodium salt (15 mg/mL, 100 L). After 5 min, mice were placed in a supine position in a chemiluminescence imaging system for imaging (with an exposure time of 5 min). Fluorescence signals were observed on days 10, 14, 21, and 28, respectively. FIG. 10 shows validation results of Luciferase expression in mouse muscle following injection with the lgAAV-CMV-Luci-7.6kb.

As shown in FIG. 10, no fluorescence was observed when positive or negative lgAAV-CMV-Luci-7.6kb vectors were injected alone, while strong fluorescence was detected with co-injection of positive or negative vectors. This demonstrated that after partial base pairing between the complementary regions of the positive and negative DNA strands, DNA synthesis was initiated from the 3′ end using the paired region as a template. This process resulted in the formation of a complete double-stranded DNA, reconstructing the full-length Luci expression cassette. This facilitated transcription of the target gene Luci, Luciferase synthesis, catalyzation of the oxidation of substrate cleavage, and light emission.

The genomes of the positive and negative vectors were 4.7 kb single-stranded, single-polarity DNA without a 3′ ITR. Therefore, neither the positive vector alone nor the negative vector alone can synthesize a second complementary DNA strand in the host cells, thus preventing gene expression (i.e., no gene expression). However, conventional rAAV-CMV-Luci shows detectable gene expression.

Collectively, the 4.7 kb single-stranded, single-polarity DNA packaged by AAV (when the positive vector and negative vector are both injected into the host cells) was able to form complementary pairing through a 1.8 kb homologous sequence inside host cells, which subsequently enabled the synthesis of a full-length 7.6 kb DNA expression cassette, leading to luciferase protein expression.

7.3 Characterization of Gene Expression in HEK 293T Cells Transfected with the lgAAV-spCAS9-gRNA-NFkB-7.0kb

### 1) Cell Infection

HEK 293T cells were seeded in 24-well plates at 2×10⁵ cells/well. After 24 h, the culture medium was removed. Viral suspension (MOI 5×10⁶) was mixed with 500 μL complete medium and then added to the well. Cells were cultured for 6 days with daily medium replacement. Cells that were not exposed to the virus served as controls.

### 2) Genomic DNA Extraction

Genomic DNA was extracted using the Blood/Cell/Tissue Genomic DNA Extraction Kit (TIANGEN BIOTECH (BEIJING) CO., LTD). Adherent cells were resuspended, centrifuged at 10,000 rpm (~11,200×g) for 1 min, and the supernatant was discarded. 200 μL buffer GA was added and vortexed until fully suspended. 20 μL Proteinase K solution was added and mixed. 200 μL buffer GB was added, mixed by inversion, and incubated at 70 °C for 10 min, until a clear solution was obtained. Brief centrifugation was performed to remove droplets on the inner wall of the tube cap. 200 μL anhydrous ethanol was added and vortexed for 15 sec. At this point, flocculent precipitate may form, and brief centrifugation was performed to remove droplets on the inner wall of the tube cap. The mixture (including the flocculent precipitate) was loaded onto the adsorption column CB3 (placed in a collection tube) and centrifuged at 12,000 rpm (~13,400×g) for 30 sec. Flow-through was discarded, and column CB3 was returned to the collection tube. 500 μL buffer GD was added, followed by centrifugation at 12,000 rpm for 30 sec, and the flow-through was discarded. Column CB3 was returned to the collection tube. 600 μL wash buffer PW was added, followed by centrifugation at 12,000 rpm for 30 sec; the flow-through was discarded, and column CB3 was returned to the collection tube. This washing step was repeated once. After column CB3 was returned to the collection tube, centrifugation was performed at 12,000 rpm (~13,400×g) for 2 min, and the flow-through was discarded. Column CB3 was air-dried at room temperature for several minutes to thoroughly remove the residual wash buffer. The column was transferred to a clean centrifuge tube, and 50 μL elution buffer TE was added dropwise to the center of the membrane. After 2-5 min incubation at room temperature, centrifugation was performed at 12,000 rpm (~13,400×g) for 2 min. The eluted DNA was collected into a centrifuge tube.

### 3) Amplification of Genomic Fragment Containing Target Sites

Nested PCR was performed as follows: For the first round, the 20 μL reaction system contained 10 μL of 2×KOD One PCR Master Mix, 200 ng of genomic DNA template, 0.5 L of each forward and reverse primer (10 μM), with the final volume adjusted to 20 μL using ddH₂O. For the second round, the 50 μL reaction system was prepared with 0.2 μL of the first-round PCR product as template, and contained 25 μL of 2×KOD One PCR Master Mix, 1.5 μL of each forward and reverse primer (10 μM), with the final volume adjusted to 50 μL using ddH₂O. The amplification conditions were as follows: pre-denaturation at 98 °C for 20 s; followed by 30 cycles of denaturation at 98 °C for 5 s, annealing at 60 °C for 10 s, and extension at 68 °C for 10 s. The target products from the second-round PCR were purified by gel extraction.

Primers Used:
NFkB2-F1:
(SEQ ID NO: 25)
5'-CCTGGGTGGTCTTCCCTGATC-3'
NFkB2-R1:
(SEQ ID NO: 26)
5'-GCACCTTGTCACAAAGCAGATAAAC-3'
NFkB2-F2:
(SEQ ID NO: 27)
5'-TGATCACAATGCTACTATGCCCTTGG-3'
NFkB2-R2:
(SEQ ID NO: 28)
5'-CTGCTGTCTTGTCCATTCGAGAAATC-3'

Synthesized by Suzhou Genewiz Biotechnology Co., Ltd.

### 4) Annealing and Enzyme Digestion

Reaction mixture was prepared from 300 ng PCR-purified product, 2 μL 10×NEBuffer 2, with the final volume adjusted to 19.7 μL using sterile water. The mixture was incubated at 95 °C water bath for 5 min, then cooled to room temperature. 0.3 μL T7 Endonuclease I was added, followed by digestion at 37 °C for 1 h. Detection was performed by 1.5% agarose gel electrophoresis.

FIG. 11 shows functional validation results of the lgAAV-spCAS9-gRNA-NFκB-7.0kb recombinant adeno-associated virus vector transduced in HEK293 cells, in which the vector expresses the target gene spCAS9, and gRNA mediated editing of the NFκB gene, wherein MW represents DNA molecular weight standard, Lane 1 corresponds to rAAV-CMV-EGFP, Lane 2 corresponds to lgAAV-spCAS9-gRNA-NF kB-7.0kb from Example 5.

FIG. 11 demonstrated insertions and deletions (indel) mutations occurred in the NFkB gene after infection with lgAAV-spCAS9-gRNA-NF kB-7.0kb recombinant adeno-associated virus vector, confirming spCas9 was functionally active.

The DNA genome of lgAAV-spCAS9-gRNA-NF k B-7.0kb was 4.7 kb single-stranded, single-polarity DNA lacking a 3′ ITR; hence, neither the positive vector alone nor the negative vector alone can synthesize a second complementary DNA strand within host cells. When the positive vector and negative vector are co-injected, although the positive and negative DNA strands exhibit only partial complementarity sequences, after partial base pairing occurs between the positive and negative DNA strands, DNA synthesis is initiated from the 3′ end, using the paired DNA as a template to generate the complementary strand, forming a complete double-stranded DNA. This reconstructs a full-length, large-gene expression cassette, enabling transcription of the large gene, protein synthesis, and exhibiting gene-editing activity. Collectively, these results demonstrated that the 4.7 kb single-stranded single-polarity DNA packaged in lgAAV could form complementary pairing through a 2.4 kb homologous DNA sequence within host cells, synthesizing a full-length 7.0 kb DNA expression cassette and expressing spCas9 protein and gRNA.

The above embodiments were provided solely for the purpose of illustrating the principles and effectiveness of the present disclosure, and were not intended to limit its scope. Any person skilled in the art could make modifications or variations to the above embodiments without departing from the scope of the present disclosure. Therefore, all equivalent modifications or changes made by those of ordinary skill in the art, based on the technical solutions disclosed herein, shall fall within the scope of the claims of the present disclosure.

1. A construction system of a recombinant adeno-associated virus vector for large gene packaging and expression, wherein the construction system is capable of packaging and expressing a DNA with a length exceeding 4.7 kb;
wherein the construction system comprises a first packaging and expression vector and a second packaging and expression vector;
wherein from 5′ end to 3′ end, the first packaging and expression vector comprises a first ITR element or an analogous element thereof and a first expression cassette, and the first expression cassette comprises a promoter, a target gene, and a polyadenylation (polyA) signal of a positive strand;
wherein from 5′ end to 3′ end, the second packaging and expression vector comprises a second ITR element or an analogous element thereof and a second expression cassette, and the second expression cassette comprises a polyA signal, a target gene, and a promoter of a negative strand;
wherein the positive strand and negative strand undergo complementary base pairing.

2. The construction system of claim 1, wherein the first ITR element or the analogous element thereof and the second ITR element or the analogous element thereof are selected from AAV viral ITR element, DttRR element, DtrsA′B′ element;
wherein the DttRR element comprises a Rep binding element (RBE) and a Rep terminal resolution site (trs) of an AAV ITR element;
wherein the DtrsA′B′ element comprises a D sequence, trs sequence, A′ sequence, and B′ sequence of the AAV ITR element.

3. The construction system of claim 1, wherein:
the AAV ITR element has a nucleotide sequence of SEQ ID NO:32;
and/or the DttRR element has a nucleotide sequence of SEQ ID NO:33;
and/or the DtrsA′B′ element has a nucleotide sequence of SEQ ID NO:34.

4. The construction system of claim 1, wherein the promoter is selected from CMV, CAG, Ef1-α, hU6, and other gene expression promoters.

5. The construction system of claim 1, wherein the large gene is one or more of modified/enhanced EGFP, modified/enhanced luciferase, and
modified/enhanced CRISPR/Cas9;
wherein the CRISPR/Cas9 is spCas9.

6. The construction system of claim 1, wherein the positive strand of the first packaging and expression vector and the negative strand of the second packaging and expression vector undergo partially complementary base pairing at 3′ end.

7. A recombinant adeno-associated virus vector system, comprising:
a first recombinant adeno-associated virus vector, wherein the first recombinant adeno-associated virus vector comprises the first packaging and expression vector of claim 1; and
a second recombinant adeno-associated virus vector, wherein the second recombinant adeno-associated virus vector comprises the second packaging and expression vector of claim 1.

8. A host cell, wherein the host cell is transduced with the recombinant adeno-associated virus vector system of claim 7.

9. The host cell of claim 8, wherein the host cell is human-derived or from other mammals.

10. A vaccine, comprising the construction system of claim 1 or a recombinant adeno-associated virus vector system comprising a first recombinant adeno-associated virus vector comprising the first packaging and expression vector of claim 1 and a second recombinant adeno-associated virus vector comprising the second packaging and expression vector of claim 1.

11. A gene therapy drug, comprising the construction system of claim 1, or a recombinant adeno-associated virus vector system comprising a first recombinant adeno-associated virus vector comprising the first packaging and expression vector of claim 1 and a second recombinant adeno-associated virus vector comprising the second packaging and expression vector of claim 1.

12. A gene editing method, comprising using administering to the subject in need a therapeutically effective amount of the construction system of claim 1 or a recombinant adeno-associated virus vector system comprising a first recombinant adeno-associated virus vector comprising the first packaging and expression vector of claim 1 and a second recombinant adeno-associated virus vector comprising the second packaging and expression vector of claim 1.

13. A genetic recombination method, comprising administering to the subject in need a therapeutically effective amount of the construction system of claim 1 or a recombinant adeno-associated virus vector system comprising a first recombinant adeno-associated virus vector comprising the first packaging and expression vector of claim 1 and a second recombinant adeno-associated virus vector comprising the second packaging and expression vector of claim 1.
